(19) United States
(12) Patent Application Publication
(10) Pub. No.: US 2019/0017045 A1
(43) Pub. Date: Jan. 17, 2019

(54) METHODS AND COMPOSITION FOR THE PRODUCTION OF SEQUENCE SPECIFIC ANTIMICROBIALS

(71) Applicants: Keun Seok Seo, Starkville, MS (US); Joo Youn Park, Starkville, MS (US)

(72) Inventors: Keun Seok Seo, Starkville, MS (US); Joo Youn Park, Starkville, MS (US)

(73) Assignee: Mississippi State University, Starkville, MS (US)

(21) Appl. No.: 16/034,089

(22) Filed: Jul. 12, 2018

Related U.S. Application Data
(60) Provisional application No. 62/531,394, filed on Jul. 12, 2017.
(60) Provisional application No. 62/534,285, filed on Jul. 19, 2017.

(51) Int. Cl. C12N 15/11; C12N 9/22; A61K 9/00; A01N 63/02; C12N 15/66; C12N 15/74; A61K 38/46; A61K 31/7088

(52) U.S. Cl. CPC C12N 15/11; C12N 9/22; A61K 9/0014; A01N 63/02; C12N 2310/20; C12N 15/74; A61K 38/465; A61K 31/7088; C12N 2800/80; C12N 15/66

(57) ABSTRACT

A method and composition for the production of sequence specific antimicrobials capable of overcoming inefficient delivery, narrow host range, and potential transfer of virulence genes by generalized transduction of phage-based delivery systems by integrating CRISPR/Cas9 system in the phage genome, removing major virulence genes from host chromosome, and expanding host specificity of phage by complementing tail fiber protein which significantly improves the efficacy and safety of CRISPR/Cas9 antimicrobials as alternative therapeutics. The method and composition provide an efficacious and safe CRISPR/Cas9 antimicrobial, broadly applicable to MRSA.

### CROSS-REFERENCE TO RELATED APPLICATIONS

This application is claims priority to U.S. Provisional Application Ser. No. 62,531,394, filed Jul. 12, 2017, and U.S. Provisional Application Ser. No. 62/534,285, filed Jul. 19, 2017, the disclosure of both are hereby incorporated by reference in their entirety.

### GOVERNMENT SUPPORT STATEMENT

This invention was made with government support under grant 1P20GM103646-01A1 awarded by the Center for Biomedical Research Excellence in Pathogen-Host interactions, National Institute of General Medical Sciences, NIH. The government has certain rights in the invention. This work was also partially supported by a grant from Animal and Plant Quarantine Agency, South Korea.

### SEQUENCE LISTING

The instant application contains a Sequence Listing which has been filed electronically in ASCII format and is hereby incorporated by reference in its entirety. Said ASCII copy, created on Jul. 12, 2018, is named 028186_175533_SL.txt and is 27,376 bytes in size.

### BACKGROUND OF THE INVENTION

Invasive infections with methicillin resistant Staphylococcus aureus (MRSA) in both community and healthcare settings totaled approximately 80,000 and accounted for 11,285 deaths in 2011, resulting in direct heath care costs of more than $4.5 billion in the United States alone (Suaya et al., 2014, Klevens et al., 2007). Moreover, increasing occurrence of vancomycin-intermediate S. aureus (reduced efficacy of vancomycin) resulted from an accumulation of single nucleotide polymorphisms in the S. aureus chromosome by long-term exposure to vancomycin (Hafer et al., 2012, Levine, 2008, Weigel et al., 2003). The increasing frequency of this problem underlines an urgent need for new antibiotics. However, the numbers of newly developed antibiotics and commercial interest in such drugs are decreasing, due to the high costs in development and rapidly rising resistance (Brown et al., 2016). These impediments have led to an interest in the development of alternative therapeutics such as vaccines, probiotics, and phage therapy that are less likely to drive resistance.

The CRISPR (Clustered, Regularly Interspaced, Short Palindromic Repeats) and CRISPR associated (Cas) genes serve as a bacterial immune system to resist foreign DNA (Sorek et al., 2013, Barrangou et al., 2007). The Cas9 present in the Type II CRISPR/Cas system of Streptococcus pyogenes is a RNA-guided endonuclease that introduces double-stranded breaks into target genes (Mali et al., 2013). The specificity of Cas9 is guided by a trans-activating small RNA (tracrRNA) and CRISPR RNA (crRNA) harboring a short spacer sequence recognizing the target gene (Semenova et al., 2011, Mojica et al., 2009). Recent studies demonstrated that a plasmid or phagemid harboring a CRISPR/Cas9 system programmed to target an antibiotic resistant gene or a specific pathogen could be delivered by a temperate phage and could successfully control antibiotic resistant Escherichia coli or MRSA with minimal effects on non-targeted bacteria (Citorik et al., 2014, Yosef et al., 2015, Bikard et al., 2014, Gomaa et al., 2014, Jiang et al., 2013). These studies demonstrated the potential use of CRISPR/Cas9 system as a programmable antimicrobial to selectively control the target bacteria at the DNA level without disturbing the normal microbiome (Citorik et al., 2014, Yosef et al., 2015, Bikard et al., 2014, Gomaa et al., 2014, Jiang et al., 2013). However, the efficacy of CRISPR/Cas9 antimicrobials is still far from being therapeutic, mainly due to the low efficiency in phage-based delivery system which limited the efficacy of CRISPR/Cas9 for reducing bacterial colony forming units (CFU) by only one or two logs in in vivo and in vitro assays (Citorik et al., 2014, Bikard et al., 2014). Furthermore, phage-based delivery systems may deliver not only a plasmid or phagemid harboring CRISPR/Cas system, but also host chromosomal segments by generalized and specialized transduction to target cells (Penades et al., 2015). This is particularly important for phage-based delivery systems using S. aureus since many important staphylococcal virulence factors such as superantigens and cytolysins are commonly located in mobile genetic elements (MGEs) and are transferred to other S. aureus and Listeria monocytogenes by generalized transduction mediated by temperate phages (Ubeda et al., 2005, Chen et al., 2009). This raises legal concerns about the regulatory compliance in therapeutic application of the phage (Loc-Carrillo et al., 2011, Bakhshinejad et al., 2014, Pirnay et al., 2015).

### BRIEF SUMMARY OF THE INVENTION

We disclose herein a genetic engineering method and composition to overcome shortcomings in phage-based delivery systems by integrating CRISPR/Cas9 system into the genome of temperate phage. Specifically, we disclose a method for the production of sequence specific antimicrobials to improve delivery to target cells comprising a pKS1 plasmid programming Cas9 nuclease to specific oligonucleotide sequence, a pKS4 plasmid integrating CRISPR/Cas9 system into the genome of a temperate phage by allelic exchange, and expanding host specificity of phage by complementing a phage tail fiber protein. We further disclose an allelic exchange method comprising using a modified pMAD-secY shuttle vector system comprising introducing a new multi-cloning site, a green fluorescent protein UV variant reporter gene (GFUuv), a chloramphenicol resistant gene (cat), and an anti-sense secY gene controlled by a tetracycline inducible promoter into a modified pMAD system, wherein the pMAD-secY system is temperature sensitive.

The aforementioned modifications improve efficiency of delivery to target cells, expand host specificity by complementing the tail fiber protein of phage, and remove virulence factor genes from the host strain to prevent contamination of harmful bacterial products in the phage lysates and spread of virulence genes by generalized transduction.

### BRIEF DESCRIPTION OF THE DRAWINGS

Further advantages of the invention will become apparent by reference to the detailed description of preferred embodiments when considered in conjunction with the drawings:

FIGS. 1A-1B depict a schematic map of the programmable and integrative CRISPR/Cas9 system. Individually, FIG. 1A depicts a programmable CRISPR/Cas9 system, synthetic oligos (SEQ ID NOS: 1 & 2) containing a promoter, pre-crRNA, and two Bbsl restriction sites flanked with a direct repeat (DR) cloned into the pMK4 shuttle vector, resulting pKS1. Underlines indicate restriction enzyme sites. Arrow indicates BbsI cleavage sites in which a spacer sequence will be cloned. FIG. 1B depicts the programmed CRISPR/Cas9 system in pKS3 specific to S. aureus, genes encoding a promoter, pre-crRNA, and spacer sequence specific to S. aureus were amplified from pKS2 and cloned into pKS3, resulting pKS4.

FIGS. 2A-2C depict Integration of CRISPR/Cas9 system into the genome of ϕSaBov lysogenized in S. aureus strain RF122. Individually, FIG. 2A depicts a bar graph showing integration of CRISPR/Cas9 system into the genome of ϕSaBov. The absolute number of transducing phage particles induced from temperate phages was determined using quantitative real time PCR and standard curves presented in FIG. 8. Bars indicate average and SEM of the number of transducing phage particles combined from triple measurements of three independent experiments (n=9). Asterisk indicates statistical significance in student t-test, compared to results from ϕ11 and 4)NM1 (p<0.001). FIG. 2B depicts a schematic map of integration of CRISPR/Cas9 system specific to S. aureus cloned in pKS4 into the non-coding region of the genome ϕSaBov between SAB1737 and SAB1738. FIG. 2C depicts integration of CRISPR/Cas9 system specific to S. aureus verified by southern blot analysis using a probe specific to the pre-crRNA gene.

FIGS. 3A-3E depict the efficacy and specificity of ϕSaBov-cas9-nuc in in vitro assays. A mid exponential culture of S. aureus strain CTH96 (1×10⁵ CFU) was treated as shown in FIG. 3A at various MOIs of ϕSaBov-Cas9-nuc or ϕSaBov-Cas9-null for 6 h or, as shown in FIG. 3B, at 50 MOI of ϕSaBov-Cas9-nuc or ϕSaBov-Cas9-null up to 24 h. Data points represent the average of triple measurements which repeated in four independent experiments. FIG. 3C depicts a mid-exponential culture of S. aureus strain CTH96pGFP or CTH96Δnuc (1×10⁵ CFU) treated at 50 MOI of ϕSaBov-Cas9-nuc for 8 h. FIG. 3D depicts a mixture of S. aureus strain CTH96pGFP and CTH96Δnuc (1:1, each at 5×10⁴ CFU) treated with ϕSaBov-Cas9-nuc at MOI of 50 for 8 h. Images showing expression of green fluorescent protein were obtained under UV wave length using Gel Doc system (Bio-Rad). FIG. 3E depicts sterile empty antibiotic disk contaminated with a suspension of CTH96 or CTH96Δint and treated with ϕSaBov-Cas9-nuc at various MOIs for 8 h. Results for FIGS. 3C-3E are a representative picture repeated in three independent experiments.

FIGS. 4A-4B. depict the efficacy of ϕSaBov-Cas9-nuc in in vivo murine skin infection. Individually, the back of C57BL/6 mouse was intradermally infected with a suspension of CTH96pGFP (1×10⁵ CFU), as shown in FIG. 4A or a mixture of CTH96pGFP and CTH96Δnuc (1:1, each at 5×10⁴ CFU), as shown in FIG. 4B, for 6 h, followed by treatment with ϕSaBov-Cas9-nuc or ϕSaBov-Cas9-null at MOI of 500. After 24 h, infected skin was excised and homogenized. Data points indicate the average of triple measurements in individual mice (n=9). Statistical significance of student t-test was indicated.

FIGS. 5A-5B. depict the prevention of toxin contamination in phage lysates. Human PBMCs were treated with phage lysates generated from RF122Δnuc or RF122-19Δnuc, an isogenic strain lacking 10 superantigen and 11 cytotoxin genes. Individually, as FIG. 5A shows a bar graph depicting, after 3 day incubation, proliferation of T cells caused by superantigens in phage lysages was measured by incorporation of radioactive ³H-thymidine into the cellular DNA using liquid scintillation counter. Bars indicate the count per minute (cpm) of radioactivity combined from triple measurements of three independent experiments (n=9). Statistical significance of student t-test was indicated. FIG. 5B depicts, after 3 h incubation, cytotoxicity by caused cytotoxins in phage lysates was measured by incorporation of propidium iodide into dead cells using flow cytometry. Data shown are representative results repeated in three independent experiments.

FIGS. 6A-6B depict expansion of host specificity of ϕSaBov. Individually, FIG. 6A shows a bar graph depicting phage spot test, phage lysates of ϕ11, ϕSaBov, or ϕSaBov-pTF11 (induced from a strain RF122-19Δnuc complemented with a plasmid containing a gene encoding the tail fiber protein of ϕ11, pTF11) inoculated onto the lawn culture of several pandemic human clones of S. aureus. FIG. 6B shows a bar graph depicting mid exponential culture of pandemic human S. aureus clones (1×10⁵ CFU) treated with phage lysates of ϕSaBov-Cas9-null, ϕSaBov-Cas9-nuc, or ϕSaBov-Cas9-nuc pTF11 (induced from a strain RF122-19ΔnucϕSaBov-Cas9-nuc complemented with pTF11) at MOI of 500 for 8 h. Bars indicate the average and SEM of Log CFU of viable cells, combined from triple measurements of three independent experiments (n=9). Asterisk indicates statistical significance in student t-test (p<0.001).

FIG. 7. depicts a schematic map of modified pMAD-secY system.

FIGS. 8A-8D depict standard curves for the absolute quantification of copy of phage DNA. Individually, FIGS. 8A, 8B, 8C and 8D depict standard curves for ϕSaBov, ϕSaBov-Cas9-nuc, ϕNM1 and ϕ11, respectively. Standard curves were generated by linear regression analysis calculating the slope, intercept, and correlation coefficient (R²) using Microcal OriginPro (Microcal origin, Version 7.5).

FIG. 9. depicts the efficacy of ϕSaBov-cas9-nuc in decolonization of S. aureus from surface of skin.

FIG. 10. depicts PCR analysis of cytolysins and superantigens genes showing the 11 cytolysin and 10 superantigen genes removed from RF122 chromosome without antibiotic selection markers by allelic exchange using modified pMAD-secY system (RF122-19).

### DETAILED DESCRIPTION

The following detailed description is presented to enable any person skilled in the art to make and use the invention. For purposes of explanation, specific details are set forth to provide a thorough understanding of the present invention. However, it will be apparent to one skilled in the art that these specific details are not required to practice the invention. Descriptions of specific applications are provided only as representative examples. Various modifications to the preferred embodiments will be readily apparent to one skilled in the art, and the general principles defined herein may be applied to other embodiments and applications without departing from the scope of the invention. The present invention is not intended to be limited to the embodiments shown, but is to be accorded the widest possible scope consistent with the principles and features disclosed herein.

Conventional antibiotics, targeting proteins of bacterial critical cellular pathways, are often rendered ineffective due to bacteria either acquiring episomes harboring resistance genes or accruing spontaneous mutations in targets (Brown et al., 2016). The CRISPR/Cas9 antimicrobials have become an attractive alternative due to the advantages of sequence-specific killing without disturbing the microbiome and multiplex features of spacer sequences to simultaneous target multiple genes, thereby preventing development of resistant mutants (Beisel et al., 2014). Despite promising results, a therapeutic use of CRISPR/Cas9 antimicrobials are still far from being practical due to the shortcomings in efficiency of delivery and safety aspects of phage-based delivery systems (Citorik et al., 2014, Bikard et al., 2014). We disclose herein a genetic engineering method and composition to enhance the efficacy and safety of phage-based delivery systems by integrating CRISPR/Cas9 system into the genome of a temperate phage to improve the delivery to target cells, complementing phage tail fiber protein to extend the host spectrum, and removing virulence genes from the host strain to prevent contamination by toxins and spread of virulence genes. In one aspect of this invention, we disclose a method for the production of sequence specific antimicrobials to improve delivery to target cells comprising a pKS1 plasmid programming Cas9 nuclease to specific oligonucleotide sequence, a pKS4 plasmid integrating CRISPR/Cas9 system into the genome of a temperate phage by allelic exchange, and expanding host specificity of phage by complementing a phage tail fiber protein.

In yet another aspect of this invention, we disclose an allelic exchange method comprising using a modified pMAD-secY shuttle vector system comprising introducing a new multi-cloning site, a green fluorescent protein UV variant reporter gene (GFUuv), a chloramphenicol resistant gene (cat), and an anti-sense secY gene controlled by a tetracycline inducible promoter into a modified pMAD system, wherein the pMAD-secY system is temperature sensitive. The aforementioned modifications improve efficiency of delivery to target cells, expand host specificity by complementing the tail fiber protein of phage, and remove virulence factor genes from the host strain to prevent contamination of harmful bacterial products in the phage lysates and spread of virulence genes by generalized transduction.

The ϕSaBov lysogenized in S. aureus strain RF122 was chosen as a candidate for phage-based CRISPR/Cas9 delivery system because induction of ϕSaBov from the strain RF122 generated an exceptionally high number of transducing phage particles harboring the phage genome. Interestingly, efficient phage genome packaging events were conserved in the RF122 background, but not reproduced when ϕSaBov was lysogenized in RN4220 or MW2 (Moon et al., 2016). These results suggest the presence of genetic elements uniquely present in the chromosome of RF122 promoting phage DNA excision and replication. Phage DNA excision, replication, and packaging are controlled by complex mechanisms involving multiple factors encoded in the phage genome and host chromosome. Upon induction of phage by SOS signals, phage-encoded rinA and rinB activate transcription of phage-encoded integrase (Int), excisionase (Xis), and unknown host encoded factors such as IHF and Fis to initiate site-specific recombination at the attachment site (att site) (Abremski et al., 1982, Ball et al., 1991, landolo et al., 2002). Genome sequence comparison of RF122, MW2, and RN4220 revealed several unique integrases, transposases, and integrative and conjugative elements associated with MGEs and reminiscent of inactivated phage present in the chromosome of RF122 (Herron-Olson et al., 2007). Thus, unique genetic elements present in RF122 play a role on efficient phage DNA excision and packaging events by ϕSaBov.

Since intravenous administration of CRISPR/Cas antimicrobials delivered by phage lysates may evoke immune responses by transducing phage particles or bacterial products remaining in the phage lysates. Moreover, repetitive administration may induce adaptive immune response resulting in antibody production, decreasing efficacy, and potential allergic reactions. Therefore, most practical application of CRISPR/Cas antimicrobials delivered by phage lysates would be topical application to the infected tissues or the contaminated surface of medical and culinary devices and food products. Recently the United States Food and Drug Administration approved phage cocktails against Listeria monocytogenes for use in ready to eat food as generally recognized as safe, further encouraging topical applications (Perera et al., 2015).

The method disclosed herein demonstrates that integration of CRISPR/Cas9 system into the phage genome significantly enhances the efficacy of S. aureus specific-killing effect by ϕSaBov-Cas9-nuc near to complete decolonization in vitro under both nutritionally enriched and limited conditions. Reductions of more than two orders of magnitude CFU were seen in an in vivo murine skin infection challenge, primarily due to the improved packaging and delivery of CRISPR/Cas9 system to target cells. However, the ϕSaBov-Cas9-nuc was unable to decolonize S. aureus from the surface of skin. It was noticed that the surface of skin was completely dried in 15 mins from the application of inoculum or phage solution that may create an environment with limited water activity which suppress transcriptional and translational activities of S. aureus, thereby the machinery of CRISPR/Cas antimicrobials could not be expressed. These results suggest that topical application of CRISPR/Cas antimicrobials to objects under dried condition will require carrier materials to increase moisture, such as hydrogel or ointment to support water activity.

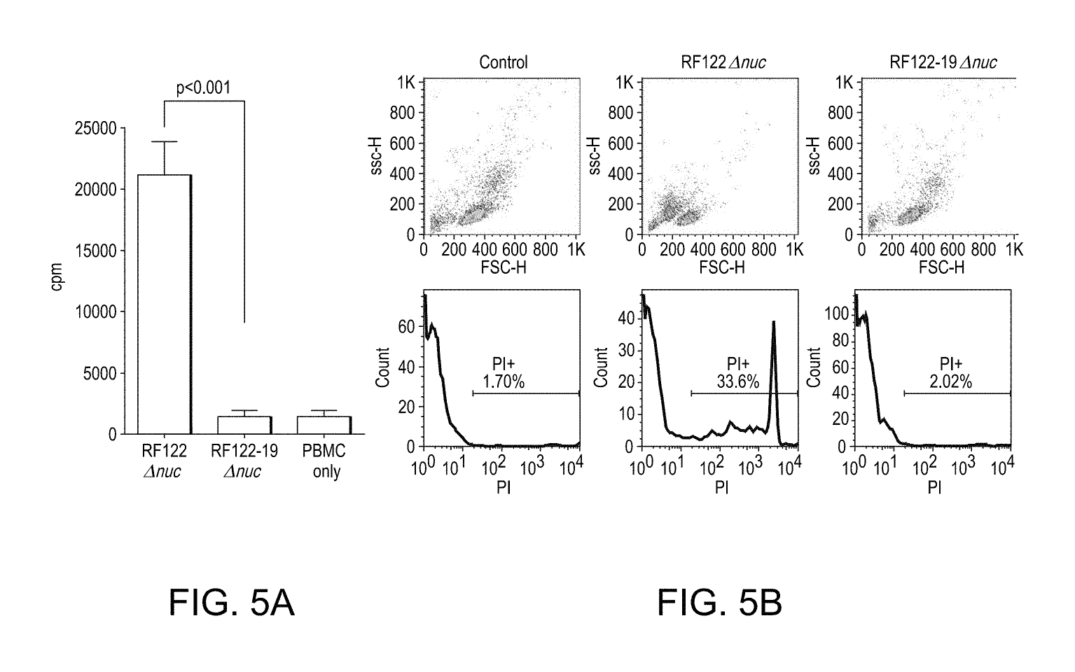

Phage lysates generated by induction or propagation of temperate phage to the host strain harboring a plasmid or phagemid containing CRISPR/Cas system are mixtures of bacterial components including bacterial DNA, proteins, and cell wall components, as well as transducing phage particles. As demonstrated in FIG. 5, phage lysates generated from the strain RF122Δnuc contained superantigens and cytolysins expressed from the chromosome of RF122. These aspects will clearly raise a regulatory compliance concern on pharmaceutical use of phage lysates containing CRISPR/Cas antimicrobials in Western clinical settings. To alleviate this concern, we generated RF122 containing multiple toxin gene deletions resulting in loss of 10 superantigen and 11 cytolysin genes (RF122-19Δnuc) by using the modified pMAD-secY temperature sensitive shuttle vector system. Therefore phage lysates generated from RF122-19Δnuc did not show any harmful effects associated with superantigens and cytolysins. Furthermore, RF122-19Δnuc can be used as a virulence factor-free host strain to propagate ϕSaBov-Cas9-nuc, without risk of spreading superantigen and cytolysin genes by temperate phage-mediated generalized transduction. One may still argue that the virulence factor-free host strain would not be an ultimate solution because phage lysates generated from RF122-19Δnuc may still contain other uncharacterized virulence factors in host chromosomal segments. My laboratory's previous study showed that excision and packaging of host chromosomal segments by ϕSaBov was highly specific to MGEs within the strain RF122 background, and not in other S. aureus strain background such as RN4220 and MW2 (Moon et al., 2016). It has also been shown that the integrase and terminase small subunit (TerS) encoded in Staphylococcus aureus pathogenicity islands (SaPI) induced sequence-specific excision of host chromosome unlinked to phage DNA which packaged into transducing phage particles by the terminase large unit (TerL) encoded in the helper phage (Chen et al., 2015). Genome sequence analysis of strain RF122 showed a single copy of the terL gene associated with the genome of ϕSaBov, and two copies of the terS genes, one with ϕSaBov and the other with SaPIbov1 (Herron-Olson et al., 2007). This invention contemplates removing the redundant integrases and TerS gene to prevent excision of host chromosome mediating generalized transduction by ϕSaBov.

Phage absorption, an important process that determines the host specificity of phage, is initiated by interaction of the phage tail fiber protein with host specific receptors such as lipopolysaccharides or the outer membrane porin protein C (Bartual et al., 2010, Winstel et al., 2013). This contact triggers conformational changes in the baseplate protein of phage, causing irreversible binding of tail fibers to the outer core of lipopolysaccharides and penetration of inner tail tube to bacterial membrane allowing ejection of phage DNA (Bartual et al., 2010). A recent study in Pseudomonas aeruginosa Pap1 phage demonstrated that a single nucleotide mutation in phage tail fiber protein resulted in altered host specificity (Le et al., 2013). Thus, host specificity of phage could be modulated by altering the phage tail fiber protein. However, parallel knowledge in S. aureus temperate phages has not been established. This method demonstrates expansion of host specificity of ϕSaBov by complementing a plasmid harboring the Tif gene of ϕ11, a broad host spectrum phage. Although not as efficacious as to CC151, the improved killing effect by ϕSaBov-Cas9-nuc pTFϕ11 against human pandemic clones was demonstrated. The partial improvement might be due to the competition of the tail fiber proteins produced by both ϕSaBov and a complemented plasmid. The complete replacement of the tif gene within ϕSaBov genome with the tif gene of broad host range phages, such as ϕ11, ϕ13, and ϕNM1 by allelic exchange is being carried out based on these findings.

A phage therapy carrying CRISPR/Cas antimicrobials undoubtedly has great potential for alternative therapeutics, supplemental to conventional antibiotics, and prophylactic measurement against increasing antibiotic resistant pathogens. The genetic engineering strategy on both phage and host genome taught herein will be useful to create an efficacious and safe CRISPR/Cas9 antimicrobials platform broadly applicable to MRSA and other important pathogens.

Development of Programmable and Integrative CRISPR/Cas9 Plasmid Vector Systmes.

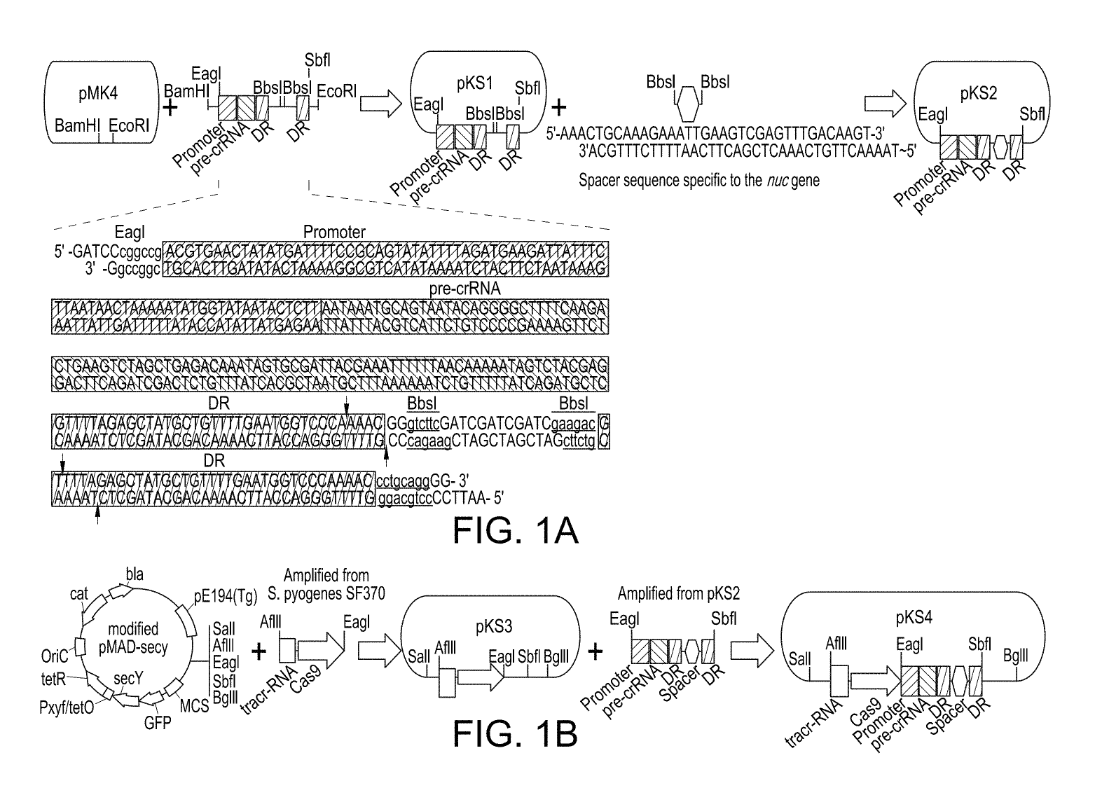

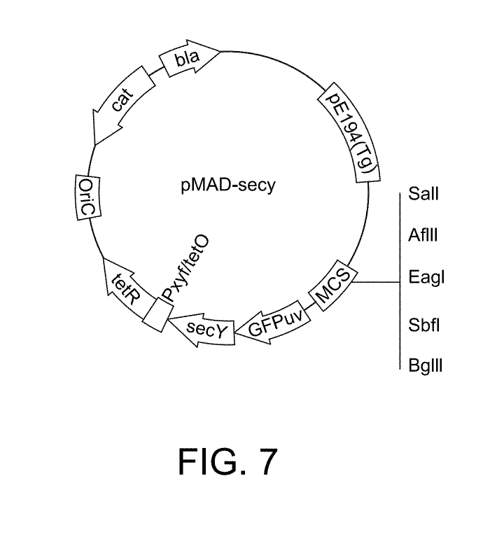

Bacteriophages can package their own genome more efficiently than host genetic elements, such as plasmids and phagemids, was the inspiration to develop a programmable and integrative vector system containing CRISPR/Cas9 integrated within the phage genome. This method was designed to improve packaging and delivery of the CRISPR system to target cells. To generate a programmable CRISPR/Cas system, synthetic oligos containing a CRISPR array encoding promoter, pre-crRNA, and direct repeats interspaced with two BbsI restriction sites was cloned into pMK4, resulting pKS1, as shown in FIG. 1A. Synthetic oligos (SEQ ID NOS: 3 & 4) containing a spacer sequence specific to the nuc gene, uniquely present in all S. aureus, followed by a protospacer adjacent motif (PAM) NGG, was cloned into Bbsl sites in pKS1, resulting pKS2, as shown in FIG. 1A. To generate integrative CRISPR/Cas9 system, tracrRNA and Cas9 genes were amplified from CRISPR/Cas9 system of Streptococcus pyogenes and cloned into the modified pMAD-secY temperature sensitive shuttle vector system, as shown in FIG. 7, resulting pKS3, as shown in FIG. 1B. To program CRISPR/Cas9 system specific to S. aureus, CRISPR array with a spacer sequence specific to the nuc gene cloned in pKS2 was amplified by PCR and cloned into pKS3, resulting pKS4, as shown in FIG. 1B. This programmed CRISPR/Cas9 system can be integrated to the genome by allelic exchange as described below.

Integration of CRISPR/Cas9 System into the Genome of ϕSaBov Lysogenized in S. aureus Strain RF122

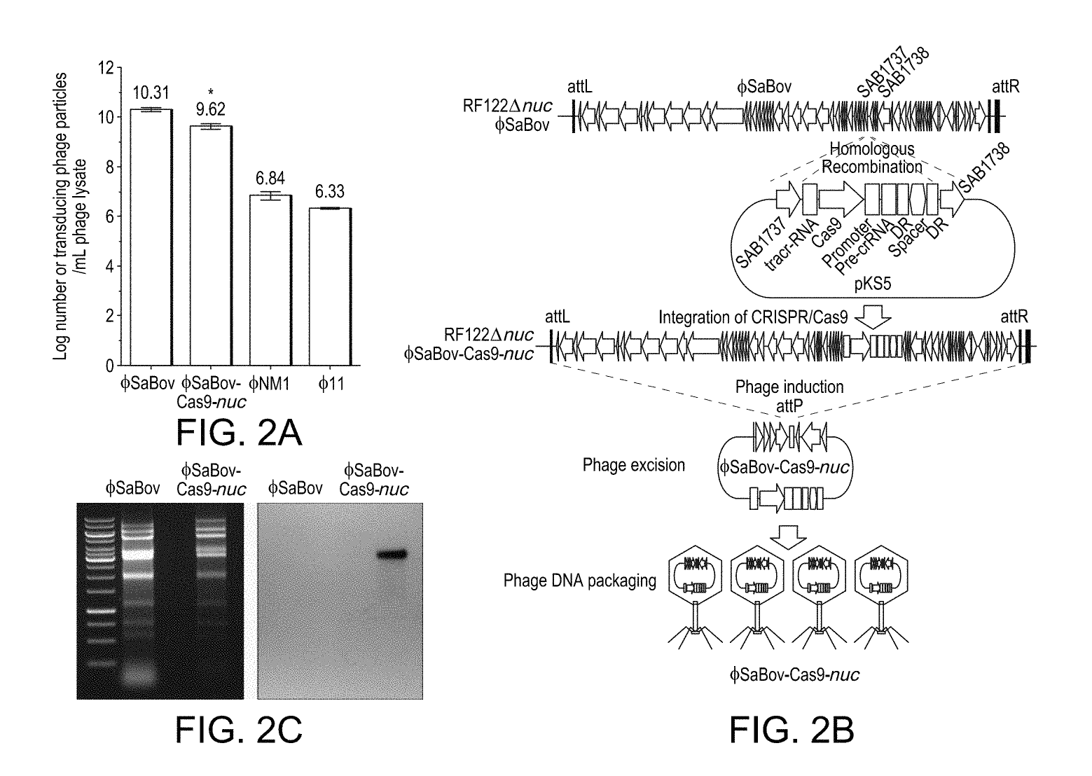

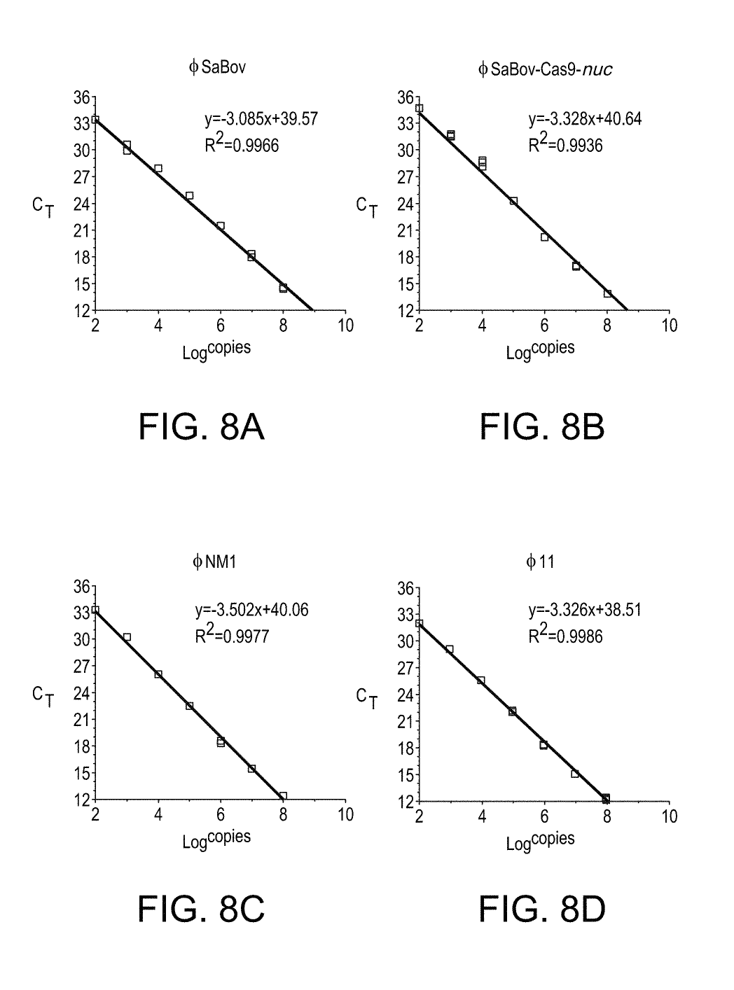

In order to select staphylococcal temperate phages efficiently packaging its own phage genome, the absolute copy number of phage DNA in the phage lysates was determined using quantitative real time PCR (qRT-PCR) and standard curves generated from serially diluted plasmid templates, as shown in FIGS. 8A-8D. For the absolute quantification of copy of phage DNA, the PCR product of the integrase gene for each phage was cloned in pCR4-TOPO. A serial dilution of cloned plasmid was used in quantitative real time PCR. Since a single transducing phage particle harbors a single copy of phage DNA, the copy number of phage DNA is equal to the number of transducing phage particles. It was found that the ϕSaBov lysogenized in S. aureus strain RF122, a bovine mastitis isolate belonging to CC151 lineage, generated exceptionally high number of transducing phage particles (10.31 Log copies/ml phage lysate) which was 3.44 and 3.98 Log magnitude higher than that of ϕ11 and ϕNM1, respectively, as shown in FIG. 2A. Based on these results, the ϕSaBov was selected for phage-based CRISPR/Cas9 delivery system.

To integrate the S. aureus-specific, programmed CRISPR/Cas9 system from pKS4 into the genome of ϕSaBov, upstream and downstream gene segments of non-coding regions between SAB1737 and SAB1738 of the ϕSaBov genome were amplified by PCR and cloned into pKS4, resulting pKS5, as shown in FIG. 2B. This plasmid was transformed into the strain RF122Δnuc ϕSaBov in which the nuc gene has been removed to prevent CRISPR/Cas9 mediated-killing. The programmed CRISPR/Cas9 system was integrated into the genome of ϕSaBov by allelic exchange, resulting in RF122Δnuc ϕSaBov-Cas9-nuc, as shown in FIG. 2B. Phages induced from this strain were referred as to ϕSaBov-Cas9-nuc. Southern blot analysis using a probe specific to the pre-crRNA gene confirmed the integration of CRISPR/Cas system into the genome of ϕSaBov, as shown in FIG. 2C. The number of transducing phage particles generated by inducing ϕSaBov-Cas9-nuc slightly decreased, compared to that by ϕSaBov, possibly due to the increase of genome size following integration of CRISPR/Cas9 system, but was still significantly higher than ϕ11 and ϕNM1, as shown in FIG. 2A. As a control, CRISPR/Cas9 system without a spacer sequence was integrated into the genome of ϕSaBov in RF122Δnuc. Phages induced from this strain were referred as to ϕSaBov-Cas9-null.

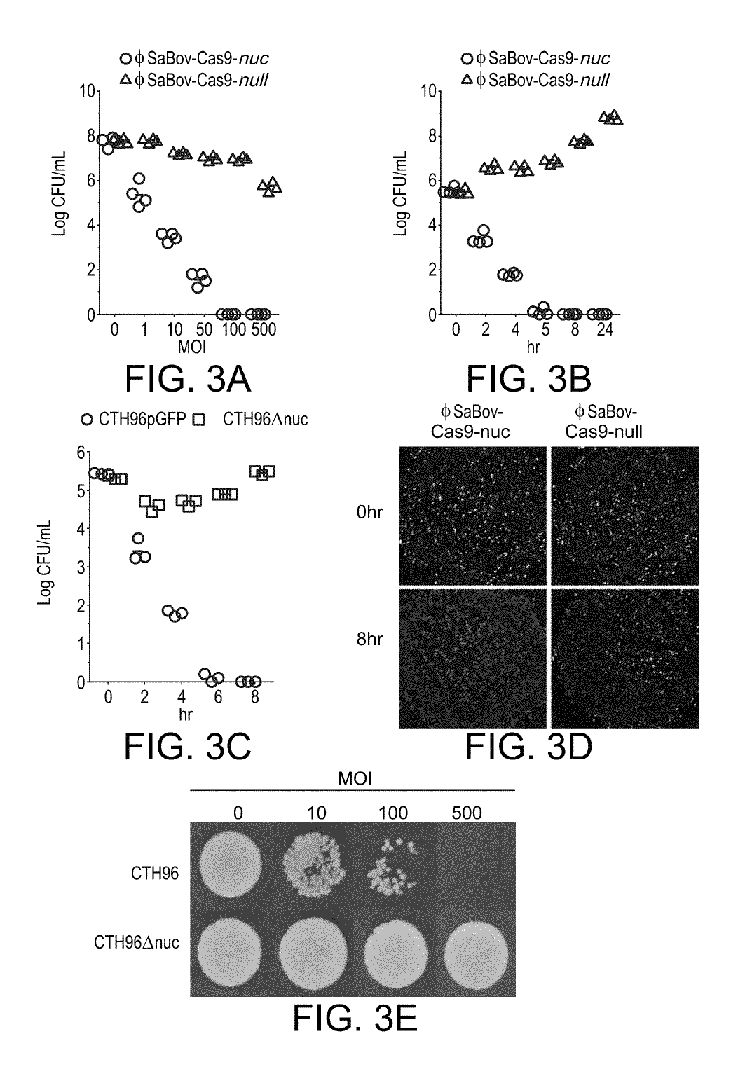

The specificity and efficacy of ϕSaBov-Cas9-nuc in in vitro assays To assess the efficacy of killing by ϕSaBov-Cas9-nuc, S. aureus strain CTH96, a bovine isolate susceptible to ϕSaBov, was treated with various multiplicities of infection (MOIs) of ϕSaBov-Cas9-nuc and viable cells were recovered by plating on BHI agar. MOI was defined as the number of transducing phage particles per recipient cell. When treated for 6 h, recipient cells were completely killed at MOI of 100 or above, and 5.01% and 0.08% of viable recipient cells were recovered at MOI of 10 and 50, respectively, as shown in FIG. 3A. In order to assess the natural occurrence of survival due to the spontaneous mutations in targets, a time-dependent killing was measured. Viable cells were recovered in BHI plates. Upon treatment with ϕSaBov-Cas9-nuc at MOI of 50, the number of viable recipient cells gradually decreased and no viable cells were recovered after 8 h treatment and sustained at 24 h, suggesting rare occurrence of surviving mutants, as shown in FIG. 3B. By contrast, treatment with ϕSaBov-Cas9-null did not show any killing effect at MOI of 50 or less and minor killing effects at MOI of 100 or above presumable due to the lytic cycle of ϕSaBov, as shown in FIGS. 3A and 3B.

To assess the nuc gene specific killing effect of ϕSaBov-Cas9-nuc, the nuc gene deletion mutant of CTH96 (CTH96Δnuc) lacking the target gene for a spacer sequence in CRISPR/Cas9 system and CTH96 carrying a plasmid expressing green fluorescent protein (CTH96pGFPuv) was generated. When treated with ϕSaBov-Cas9-nuc at a MOI of 50, viable CTH96pGFPuv gradually decreased and completely lost viability at 8 h of treatment, as shown in FIG. 3C. By contrast, the number of viable CTH96Δnuc slightly decreased within 2 h of treatment, presumable by the lytic cycle of ϕSaBov, and then gradually increased thereafter, as shown in FIG. 3C. Next, the mixed cultures of CTH96Δnuc and CTH96pGFPuv were treated with ϕSaBov-Cas9-nuc or ϕSaBov-Cas9-null at MOI of 50 for 8 h and viable cells recovered on BHI plates were analyzed under the UV lamp. When treated with the ϕSaBov-Cas9-nuc, recipient cells expressing GFP were selectively killed, as shown in FIG. 3D. By contrast, when treated with the ϕSaBov-Cas9-null, both CTH96Δnuc and CTH96pGFPuv were equally recovered. The killing effect of ϕSaBov-Cas9-nuc against S. intermedius (coagulase positive staphylococci) and S. epidermidis (coagulase negative staphylococci) was also tested but was not observed. Combined, these results clearly demonstrate the nuc gene specific killing effect of ϕSaBov-Cas9-nuc.

The effect of CRISPR/Cas9 system requires biological activities including transcription and translation of the CRISPR/Cas9 system within the recipient cells which might limit the application of CRISPR/Cas9 antimicrobials under the nutritionally and metabolically-limited conditions. To simulate the nutritionally and metabolically-limited conditions, antibiotic disks contaminated with recipient cells in PBS (1×10⁵ CFU) were treated with the ϕSaBov-Cas9-nuc at MOIs of 10, 100, and 500. After 8 h treatment, viable cells were recovered by blotting the disk on to the BHI plate. Recipient cells were gradually decreased at MOIs of 10 and 100, and completely decolonized at MOIs of 500 (FIG. 3E). By contrast, the viability of recipient cells lacking the nuc gene (CTH96Δnuc) was not affected, as shown in FIG. 3E. This invention contemplates the use of ϕSaBov-Cas9-nuc for sanitizing abiotic objects such as medical devices or surfaces.

The Efficacy of ϕSaBov-Cas9-nuc in in vivo Assays

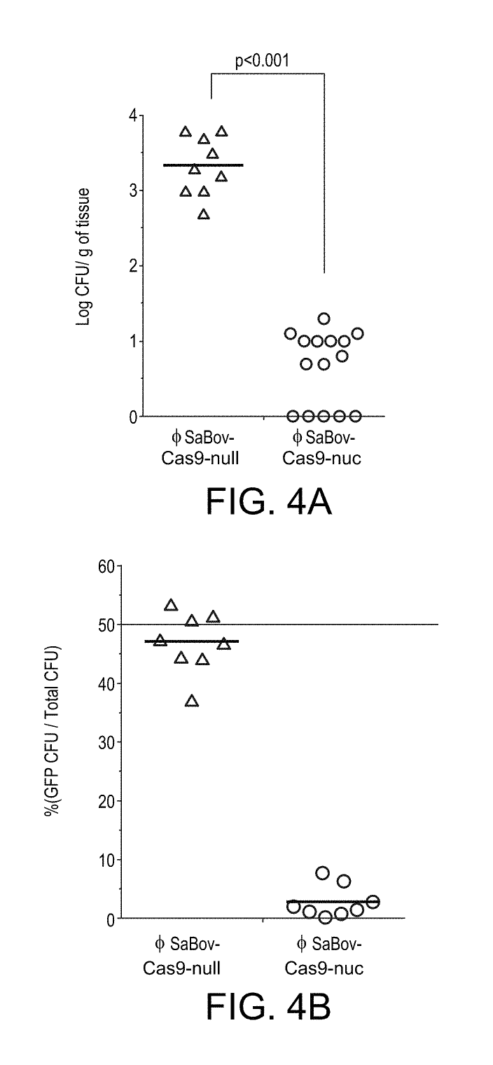

To test the efficacy of ϕSaBov-Cas9-nuc in in vivo, the back of C57BL/6 mouse was shaved and intradermally inoculated with recipient cells (CTH96pGFP, 1×10⁵ CFU). After 6 h of infection, ϕSaBov-Cas9-nuc or ϕSaBov-Cas9-null was injected to the infected skin at MOI of 500. Following treatment for 24 h, infected skins were excised and homogenized to recover viable cells from infected skins. Viable cells were recovered by plating serially diluted homogenates onto BHI plate. The specificity of ϕSaBov-Cas9-nuc was evaluated by the proportion of viable cells expressing green fluorescent protein in total viable cells. The number of viable cells recovered from the skins treated with ϕSaBov-Cas9-nuc (0.647±0.128 Log CFU/g of tissue, mean±SEM) was significantly lower than that treated with ϕSaBov-Cas9-null (3.333±0.131 Log CFU/g of tissue), as shown in FIG. 4A. Next, to test the nuc gene specific killing capacity of ϕSaBov-Cas9-nuc in in vivo, animals were infected with a mixture of CTH96pGFP and CTH96Δnuc (1:1, each at 5×10⁴ CFU) for 6 h, followed by treatment with ϕSaBov-Cas9-nuc or ϕSaBov-Cas9-null at MOI of 500 for 24 h. The proportion of viable cells expressing GFP in infected skin treated with ϕSaBov-Cas9-nuc was 2.8±2.6%, in contrast to those treated with ϕSaBov-Cas9-null showing 47.1±4.9%, as shown in FIG. 4B, suggesting nuc gene specific killing effect by ϕSaBov-Cas9-nuc in in vivo.

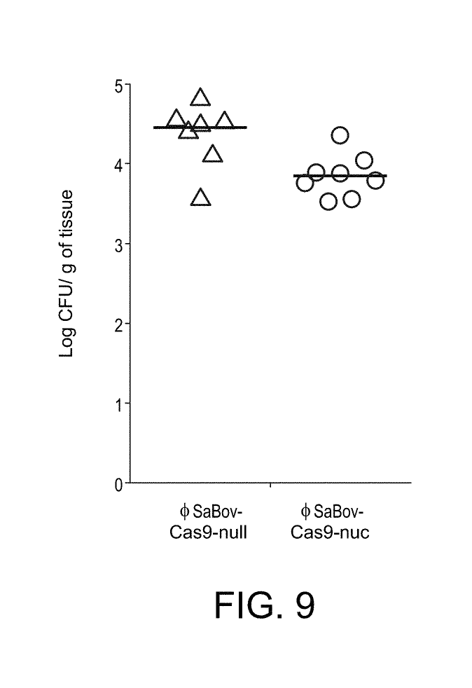

Lastly, it was tested if the ϕSaBov-Cas9-nuc were able to decolonize S. aureus from the surface of skin. The back of mice skin was shaved, depilated, decolonized with 70% alcohol, and colonized with CTH96pGFP (2×10⁴ CFU) by cotton swab. After 6 h, ϕSaBov-Cas9-nuc or ϕSaBov-Cas9-null at MOI of 500 was topically applied by spraying. Following treatment for 24 h, infected skins were dissected and homogenized to determine the viable cell count. However, the number of viable cells recovered from infected skins treated with ϕSaBov-Cas9-nuc was not significantly different from that treated with ϕSaBov-Cas9-null, as shown in FIG. 9.

Prevention of Toxins Contaminations in Phage Lystes

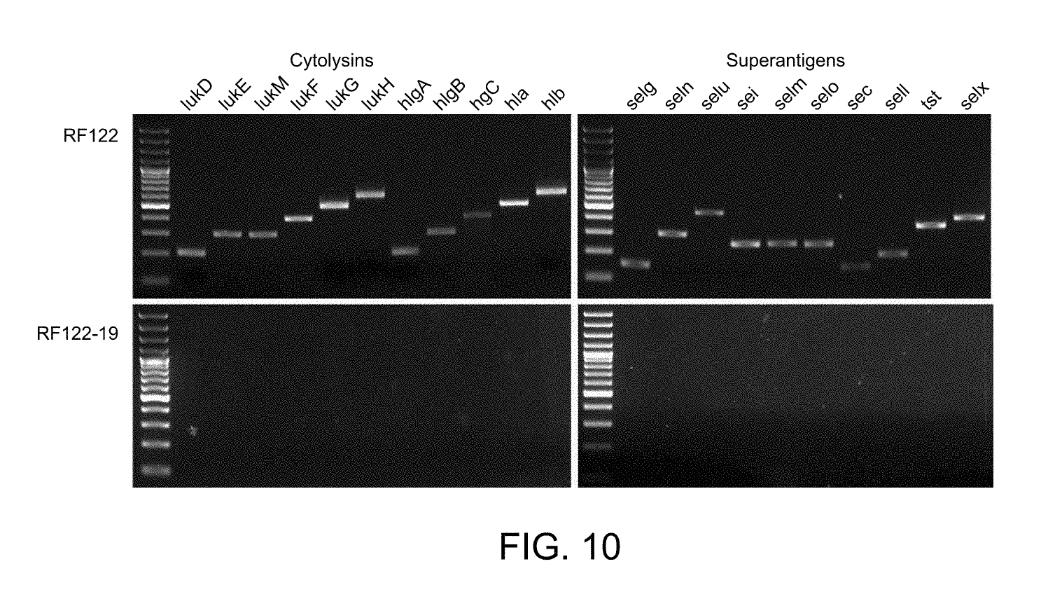

The strain RF122 harbors 10 superantigens (sec, seg, sei, selm, seln, selo, selu, sell, tstl, selx) and 11 cytolysins (hla, hlb, hlgA, hlgB, hlgC, lukD, lukE, lukG, lukH, lukM, lukF′) genes, as shown in FIG. 10 which could be contaminated in the phage lysates generated from the strain RF122. Furthermore, previous studies of my laboratory demonstrated that induction of ϕSaBov from strain RF122 generated transducing phage particles harboring genetic segments specifically associated with MGEs such as vSaα, vSaβ, vSaγ, and SaPI1 some of which contain superantigens (sec, seg, sei, sem, sen, seo, tst) and cytolysins (hla, lukD, lukE) and transferred these virulence genes to other S. aureus strains by generalized transduction (Moon et al., 2016, Moon et al., 2015). To prevent contamination of these toxins in phage lysates and potential spread of toxin genes by the ϕSaBov-based delivery system, 10 superantigens and 11 cytotoxins present in the chromosome of RF122Δnuc ϕSaBov-Cas9-nuc were removed by allelic exchange using modified pMAD-secY system, resulting RF122-19Δnuc ϕSaBov-Cas9-nuc. The deletions were confirmed by PCR analysis, as shown in FIG. 10. As expected, phage lysates generated from the strain RF122Δnuc showed superantigenicity as indicated by significant higher count per minute (cpm) as a result of incorporation of ³H-thymidine into dividing cellular DNA, as shown in FIG. 5A. Consistently, incubation with phage lysates generated from the strain RF122Δnuc induced cytotoxicity by cytolysins in phage lysates causing membrane damage allowing chelation of propidium iodide into cellular DNA, as shown in FIG. 5B. By contrast, phage lysates generated from the strain RF122-19Δnuc did not induce any superantigenicity or cytotoxicity, as shown FIG. 5A and 5B.

Expansion of Host Specificity of ϕSaBov

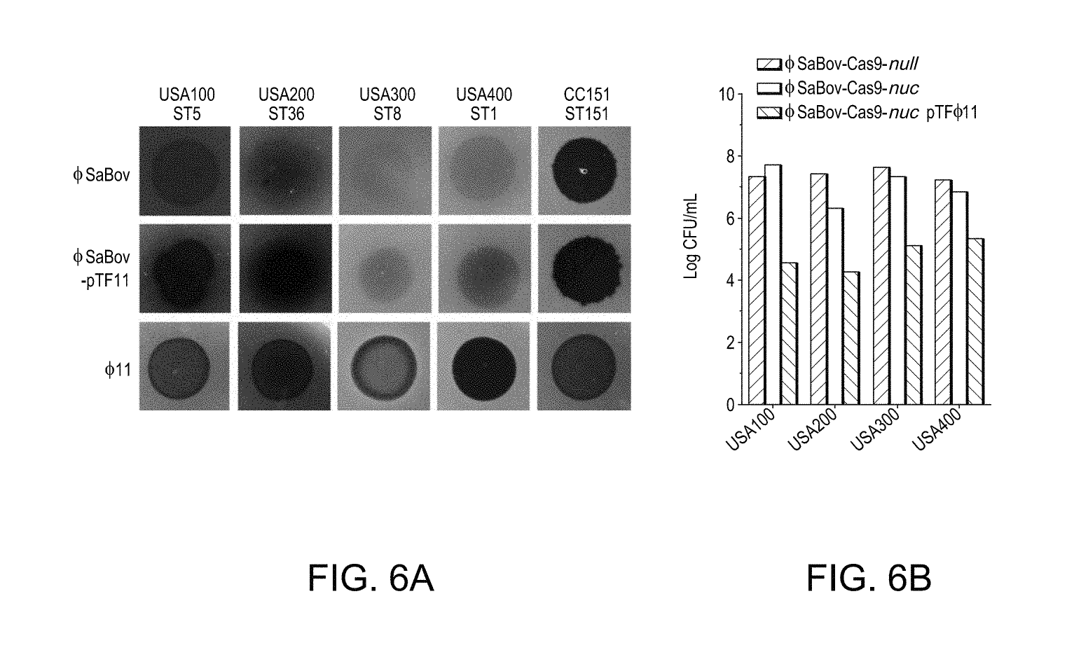

The ϕSaBov has a narrow host range highly specific to bovine CC151 lineage of S. aureus as shown in phage spot test, as shown in FIG. 6A, top panel, thereby the efficacy of ϕSaBov-Cas9-nuc to human pandemic clonal lineage of S. aureus (ST1, ST5, ST8 and ST36) was minimal or had no effect, as shown in FIG. 6B, black bars. Host specificity of phage is mainly determined by phage-encoded tail fiber protein interacting with receptors on host cells such as membrane proteins or cell wall carbohydrates (Bartual et al., 2010, Winstel et al., 2013, Le et al., 2013). It was sought to expand the host ranges of ϕSaBov by complementing the gene encoding the tail fiber protein (Tif) of ϕ11 which has a broad spectrum of host range in several pandemic clones of human S. aureus isolates in the United States, as shown in FIG. 6A, bottom panel. A pMK4 shuttle vector harboring the tail fiber protein gene of ϕ11 (pTF11) was constructed and transformed into the strain RF122-19Δnuc ϕSaBov and RF122-19Δnuc ϕSaBov-Cas9-nuc, resulting RF122-19Δnuc ϕSaBov-pTF11 and RF122-19Δnuc ϕSaBov-Cas9-nuc-pTF11, respectively. Phages induced from RF122 Δnuc ϕSaBov-pTF11 (ϕSaBov-pTF11) showed improved clear zone of lysis in phage spot test against pandemic human clonal lineage (ST1, ST5, ST8 and ST36) of S. aureus strains, as shown in FIG. 6A, middle panel, compared to that of ϕSaBov. Consistently, phages induced from RF122 Δnuc ϕSaBov-Cas9-nuc pTF11 significantly improved reduction of Log CFU against ST1, ST5, ST8 and ST36 lineages of S. aureus ranging 1.51 to 3.15 order of magnitude, as shown in FIG. 6B, gray bars.

The terms “comprising,” “including,” and “having,” as used in the claims and specification herein, shall be considered as indicating an open group that may include other elements not specified. The terms “a,” “an,” and the singular forms of words shall be taken to include the plural form of the same words, such that the terms mean that one or more of something is provided. The term “one” or “single” may be used to indicate that one and only one of something is intended. Similarly, other specific integer values, such as “two,” may be used when a specific number of things is intended. The terms “preferably,” “preferred,” “prefer,” “optionally,” “may,” and similar terms are used to indicate that an item, condition or step being referred to is an optional (not required) feature of the invention.

The invention has been described with reference to various specific and preferred embodiments and techniques. However, it should be understood that many variations and modifications may be made while remaining within the spirit and scope of the invention. It will be apparent to one of ordinary skill in the art that methods, devices, device elements, materials, procedures and techniques other than those specifically described herein can be applied to the practice of the invention as broadly disclosed herein without resort to undue experimentation. All art-known functional equivalents of methods, devices, device elements, materials, procedures and techniques described herein are intended to be encompassed by this invention. Whenever a range is disclosed, all subranges and individual values are intended to be encompassed. This invention is not to be limited by the embodiments disclosed, including any shown in the drawings or exemplified in the specification, which are given by way of example and not of limitation.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

All references throughout this application, for example patent documents including issued or granted patents or equivalents, patent application publications, and non-patent literature documents or other source material, are hereby incorporated by reference herein in their entireties, as though individually incorporated by reference, to the extent each reference is at least partially not inconsistent with the disclosure in the present application (for example, a reference that is partially inconsistent is incorporated by reference except for the partially inconsistent portion of the reference).

### Brief Description of the Sequences

Bacterial strains and growth conditions. All strains and plasmids used in this invention are listed in Table 1. Staphylococcus aureus strains were cultured in tryptic soy broth (TSB) or agar (TSA) plates (Difco) supplemented with chloramphenicol (10 μg/mL, Sigma-Aldrich) as necessary. Escherichia coli were grown in Luria-Bertani (LB) broth and agar plates supplemented with ampicillin (100 μg/mL, Sigma-Aldrich) as necessary.

TABLE 1
Bacterial strains and plasmids used in this invention.

| Strain | Description | Reference or source |
|---|---|---|
| Staphylococcus aureus | | |
| RF122 | Bovine isolate, CC151, Lysogenized with | (32) |
| | ϕSaBov | |
| RF122Δnuc | The nuc gene deletion mutant of RF122 | This invention |
| RF122Δnuc ϕSaBov-Cas9- | Integration of CRISPR-Cas9 system specific to | This invention |
| nuc | the nuc gene into the genome of ϕSaBov | |
| | lysogenized in RF122 | |
| RF122Δnuc ϕSaBov-Cas9- | Integration of CRISPR-Cas9 system without | This invention |
| null | spacer sequence into the genome of ϕSaBov | |
| | lysogenized in RF122 | |
| RF122-19 | 10 cytotoxins and 11 superantigen gene | This invention |
| | deletions mutant of RF122 | |
| RF122-19Δnuc | The nuc gene deletion mutant of RF122-19 | This invention |
| RF122-19ΔnucϕSaBov- | Integration of CRISPR-Cas9 system specific to | This invention |
| Cas9-nuc | the nuc gene into the genome of ϕSaBov | |
| | lysogenized in RF122-19 | |
| RF122-19ΔnucϕSaBov- | Integration of CRISPR-Cas9 system without | This invention |
| Cas9-null | spacer sequence into the genome of ϕSaBov | |
| | lysogenized in RF122-19 | |
| RF122-19ΔnucϕSaBov- | Complementation of ϕ11 tail fiber protein gene | This invention |
| pTF11 | in RF122-19Δnuc | |
| RF122-19ΔnucϕSaBov- | Complementation of ϕ11tail fiber protein gene | This invention |
| Cas9-nuc-pTF11 | in RF122-19ΔnucϕSaBov-Cas9-nuc | |
| CTH96 | Bovine isolate, CC151, susceptible to ϕSaBov | (37) |
| CTH96Δnuc | The nuc gene deletion mutant of CTH96 | This invention |
| CTH96pGFP | Expression of green fluorescence protein on | This invention |
| | CTH96 | |
| NRS382 | Human MRSA USA100, ST5 | (39) |
| MN PE | Human MRSA USA200, ST36 | (24) |
| DAR1809 | Human MRSA USA300, ST8 | (24) |
| MW2 | Human MRSA USA400, ST1 | (39) |
| Escherichia coli | | |
| DH5α | Cloning host of pMAD and pMK4 | Life |
| | | Technologies |
| Top10 | Cloning host of pCR4 | Life |
| | | Technologies |
| Plasmid | Description | Reference |
| Modified pMAD-secY | Temperature sensitive shuttle vector system | This invention |
| pCR4-TOPO | TA cloning vector | Life |
| | | Technologies |
| pMK4 | High copy number vector for complementary | (40) |
| pKS1 | Cloning of synthetic oligos containing a | This invention |
| | promoter, pre-crRNA, and two BbsI restriction | |
| | sites flanked with a direct repeat (CRISPR | |
| | array) into pMK4 | |
| pKS2 | Cloning of the spacer sequence specific to the | This invention |
| | nuc gene into pKS1 | |
| pKS3 | Cloning of a tracrRNA and Cas9 into modified | This invention |
| | pMAD-secY | |
| pKS4 | Cloning of PCR product containing CRISPR | This invention |
| | array with spacer sequence specific to the nuc | |
| | gene amplified from pKS2 into pKS3 | |
| pKS5 | Cloning of SAB1737 and SAB1738 into pKS4 | This invention |

Plasmid construction. All oligos used in this invention are listed in Table 2. Synthetic oligos (CRISPR_f/CRISPR_r) containing promoter, pre-crRNA, and direct repeats flanked with Bbsl sites (CRISPR array) were annealed, ligated into pMK4 digested with BamHI and EcoRI, resulting pKS1. Synthetic oligos (spacer-nucf/spacer-nucr) containing a spacer sequence specific to the nuc gene followed by protospacer-adjacent motif (NGG) were annealed and ligated into pKS1 digested with BbsI, resulting pKS2. The tracrRNA and the cas9 genes were amplified from the genomic DNA of Streptococcus pyogenes SF370 (ATCC) using oligos (tracrrnaf/cas9r), followed by digestion with AflII and EagI, and ligation into corresponding sites in modified pMAD-secY temperature sensitive shuttle vector, resulting pKS3. To program tracrRNA and the cas9 gene specific to S. aureus, CRISPR array specific to the nuc gene was amplified from pKS2 using oligos (leaderf/drr), digested with EagI and SbfI, and cloned into corresponding sites in pKS3, resulting pKS4.

TABLE 2
Oligonucleotides used in this invention

| | |
|---|---|
| Name | Sequences (5′ to 3′) |

Cloning of Cloning of CRISPR array containing a promoter, pre-crRNA.
DR, and BbsI sites

| | |
|---|---|
| CRISPR_f | SEQ ID NO: 1 |
| | GATCCCGGCCGACGTGAACTATATGATTTTCCGCAGTATA |
| | TTTTAGATGAAGATTATTTCTTAATAACTAAAAATATGGT |
| | ATAATACTCTTAATAAATGCAGTAATACAGGGGCTTTTCA |
| | AGACTGAAGTCTAGCTGAGACAAATAGTGCGATTACGAA |
| | ATTTTTTAGACAAAAATAGTCTACGAGGTTTTAGAGCTAT |
| | GCTGTTTTGAATGGTCCCAAAACGGGTCTTCGATCGATCG |
| | ATCGAAGACGTTTTAGAGCTATGCTGTTTTGAATGGTCCC |
| | AAAACCTTGCAGGGG |
| CRISPR_r | SEQ ID NO: 2 |
| | AATTCCCCTGCAGGGTTTTGGGACCATTCAAAACAGCATA |
| | GCTCTAAAACGTCTTCGATCGATCGATCGAAGACCCGTTT |
| | TGGGACCATTCAAAACAGCATAGCTCTAAAACCTCGTAGA |
| | CTATTTTTGTCTAAAAAATTTCGTAATCGCACTATTTGTCT |
| | CAGCTAGACTTCAGTCTTGAAAAGCCCCTGTATTACTGCA |
| | TTTATTAAGAGTATTATACCATATTTTTAGTTATTAAGAAA |
| | TAATCTTCATCTAAAATATACTGCGGAAAATCATATAGTT |
| | CACGTCGGCCGG |

Cloning of spacer sequencing into pKS1

| | |
|---|---|
| spacer-nucf | SEQ ID NO: 3 |
| | AAACTGCAAAGAAAATTGAAGTCGAGTTTGACAAGT |
| spacer-nucr | SEQ ID NO: 4 |
| | TAAAACTTGTCAAACTCGACTTCAATTTTCTTTGCA |

Cloning of trac-RNA and Cas9 into modified pMAD-secY

| | |
|---|---|
| Tracrmaf | SEQ ID NO: 5 GATCCTTAAGTGATCCCTTGAAAGATTCTGT |
| cas9r | SEQ ID NO: 6 |
| | GATCCGGCCGTCAGTCACCTCCTAGCTGACTCA |

Cloning of CRISPR array with a spacer sequence specific to the nuc into pKS3

| | |
|---|---|
| Leader | SEQ ID NO: 7 |
| | GCAGGTCGACGGATCCCGGCCGACGTGAACTATATGATTT |
| | TCCGC |
| drr | SEQ ID NO: 8 |
| | AACGACGGCCAGTGAATTCCCCTGCAGGGTTTTGGGACCA |
| | TTCAAAACAGCATAGCTCTAAAACGTCTTCGATCGATCGA |
| | TCGAAGACCC |

Cloning of SAB1737 and SAB1738 into pKS4

| | |
|---|---|
| 1737upf | SEQ ID NO: 9 GATCGTCGACTTATGCTTCACTCCATTTC |
| 1737upr | SEQ ID NO: 10 GATCCTTAAGATGGGCAGTGTTGTAATTAT |
| 1738dnf | SEQ ID NO: 11 |
| | GATCCCTGCAGGTGTTGTTGCATTAAATCACT |
| 1738dnr | SEQ ID NO: 12 GCGCAGATCTTGATATTTAGAGGTGGCACA |

Generating the nuc gene deletion mutant

| | |
|---|---|
| Nucupf | SEQ ID NO: 13 |
| | GATCGTCGACGTTAACACTTTAAGCAAACCGCATC |
| Nucupr | SEQ ID NO: 14 |
| | GATCACGCGTAACTAACACCTCTTTCTTTTTAG |
| Nucdnf | SEQ ID NO: 15 |
| | GATCGAATTCTGCTCATTGTAAAAGTGTCACTGCT |
| Nucdnr | SEQ ID NO: 16 GATCCCCGGGATACGTCGCTACCATCTTCT |

Generating α-hemolysin (hla) deletion mutant

| | |
|---|---|
| Hlaupf | SEQ ID NO: 17 GCGCGGATCCTTACCTCATATAGTGTCATG |
| Hlaupr | SEQ ID NO: 18 GCGCGTCGACGAAAGGTACCATTGCTGGTC |
| Hladnf | SEQ ID NO: 19 GCGCGAATTCGTCAATTTAGAATATTGCAG |
| Hladnr | SEQ ID NO: 20 GCGCAGATCTAATGCCTCTAACTAAAAACC |

Generating β-hemolysin, leukocidin G/H deletion mutant

| | |
|---|---|
| 1874f | SEQ ID NO: 21 GCGCGGATCCCTTAATTCCGATTACATTTG |
| 1874r | SEQ ID NO: 22 GCGCGTCGACGTGCCTTTATTAACATTAAG |
| 1876f | SEQ ID NO: 23 GCGCGAATTCCTTCAAGTCATTCGCAATAA |
| 1876r | SEQ ID NO: 24 GCGCAGATCTGTATCAACGATCTTATTAAC |

Generating gamma hemolysin ACB deletion mutant

| | |
|---|---|
| Hlgaupf | SEQ ID NO: 25 TAATGGATCCACCGTTGATTCTCAATCG |
| Hlgaupr | SEQ ID NO: 26 TGAAGTCGACCATCTTAACAACTAGGGC |
| Hlgbdnf | SEQ ID NO: 27 GCGCGAATTCGGCTTTGTGAAACCTAATCC |
| Hlgbdnr | SEQ ID NO: 28 GCGCAGATCTGGTCGTCACAATTACTGTTG |

Generating leukocidin D/E (lukDE′) deletion mutant

| | |
|---|---|
| Lukdeupf | SEQ ID NO: 29 GCGCGGATCCCGAATTTGGAGATGGCTGC |
| Lukdeupr | SEQ ID NO: 30 GCGCGTCGACCTAATCCTGGAGTATAACTG |
| Lukdednf | SEQ ID NO: 31 TGATGAATTCCTATTGCCCGTTAAACGG |
| Lukdenr | SEQ ID NO: 32 ATTGAGATCTCCTGTCGGTTTACTCATTG |

Generating leukocidin M/F (lukMF′) deletion mutant

| | |
|---|---|
| Lukmfupf | SEQ ID NO: 33 GCGCGGATCCTTCGTATAGGCTTTATAG |
| Lukmfupr | SEQ ID NO: 34 GCGCGTCGACCTCCAATGTTATATCCTA |
| Lukmfdnf | SEQ ID NO: 35 GCGCGAATTCCTACTTCCTAGATACCGT |
| Lukmfdnr | SEQ ID NO: 36 GCGCAGATCTGAATAGCTTAACAACGTA |

Generating Enterotoxin gene cluster (egc) deletion mutant

| | |
|---|---|
| Egcupf | SEQ ID NO: 37 TCTTGATACGTATTTGACACTTGC |
| Egcupr | SEQ ID NO: 38 AGCTATACGAGTTTGATGGTTCTG |
| egcdnf | SEQ ID NO: 39 |
| | CAGAACCATCAAACTCGTATAGCTAACTAAGCGACTCAG |
| | ATAATAGAC |
| Egcdnr | SEQ ID NO: 40 AGAGTTGTTACAGTCGCTACACC |

Generating staphylococcal enterotoxin C deletion mutant

| | |
|---|---|
| Secupf | SEQ ID NO: 41 ATGAATTCCTGTGGATTTAGAAATAAGG |
| Secupr | SEQ ID NO: 42 CCAACATTCCCAAGAAGTATC |
| secdnf | SEQ ID NO: 43 |
| | GATACTTCTTCTTGGGAATGTTGGAAGAATGGATAATGTT |
| | AATCC |
| Secdnr | SEQ ID NO: 44 TTATCCATGGCAAGCATCAAAC |

Generating staphylococcal enterotoxin like toxin L deletion mutant

| | |
|---|---|
| Selupf | SEQ ID NO: 45 GATATATTTGAAAGGTAAGTACTTCG |
| Selupr | SEQ ID NO: 46 AGTGTAGTATTCCATATGAATGATGGT |
| seldnf | SEQ ID NO: 47 |
| | ACCATCATTCATATGGAATACTACACTATACAAAAGGTTA |
| | TAGGAAGAGTT |
| Seldnr | SEQ ID NO: 48 CAATTTCTACAGATATGACTCCC |
| Dself | SEQ ID NO: 49 GTCATGTTTCGGTTGATAGG |
| Dselr | SEQ ID NO: 50 TGTACAAATGGACTTAAGATATAGCG |

Generating toxic shock syndrome toxin deletion mutant

| | |
|---|---|
| Tstupf | SEQ ID NO: 51 |
| | GCGCGGATCCACTACATGTACTTACCAATGCG |
| Tstupr | SEQ ID NO: 52 |
| | GCGCGTCGACGCAGAAATTAATTAATTTACCACTTTTTCT |
| Tstdnf | SEQ ID NO: 53 |
| | GCGCGAATTCCAAAGGGCTTACGATGAAAAATTTCAT |
| Tstdnr | SEQ ID NO: 54 |
| | GCGCAGATCTAACCAATTACCAAATTCTCCATGC |

Generating staphylococcal enterotoxin like toxin X deletion mutant

| | |
|---|---|
| Selxupf | SEQ ID NO: 55 TGTCGATGCTATGGATAGTGAGG |
| Selxupr | SEQ ID NO: 56 TAATTACCTCCTTGATGTAAAGC |
| selxdnf | SEQ ID NO: 57 |
| | GCTTTACATCAAGGAGGTAATTATATCGCTAATACTTTGA |
| | AAGTTAGG |
| Selxdnr | SEQ ID NO: 58 TCAAATGTAGCAGTATACATTAATTGCG |

Complementation of the tail fiber protein of ϕ11

| | |
|---|---|
| Synf | SEQ ID NO: 59 GCTACTGCAGATCCCATTATGCTTTGGCAG |
| Synr | SEQ ID NO: 60 GGGTTTCACTCTCCTTCTACA |
| ϕ11tailf | SEQ ID NO: 61 |
| | AGGAGAGTGAAACCCATGTACAAAATAAAAGATGTTGAA |
| | AC |
| ϕ11autor | SEQ ID NO: 62 |
| | GCTAGGATCCCTAACTGATTTCTCCCCATAAG |

Confirmation of deleted cytotoxins and superantigens

| | |
|---|---|
| Lukdf | SEQ ID NO: 63 TTGCCAGTCAACTTCATAAGTAGATGT |
| Lukdr | SEQ ID NO: 64 GCGCGTGGTAACTTTAACCC |
| Lukef | SEQ ID NO: 65 TTTTTTACCATCAGGCGTAACA |
| Luker | SEQ ID NO: 66 ACGAATGATTTGGCCATTCC |
| Lukmf | SEQ ID NO: 67 TGGAGGTAAATTCCAGTCAGCA |
| Lukmr | SEQ ID NO: 68 TGTCGCGATAAAAGACGGATT |
| lukf f | SEQ ID NO: 69 ACTGGTGGATTGAACGGGTC |
| lukf r | SEQ ID NO: 70 ATCCAGTGCAAGTTGTTCCAAA |
| Lukgf | SEQ ID NO: 71 GACTTTGCACCAAAAAATCAGGAT |
| Lukgr | SEQ ID NO: 72 AGGTGCATAATGTATTCCAGGTCT |
| lukhf | SEQ ID NO: 73 GCGTCATCATTATCATGTGCAA |
| Lukhr | SEQ ID NO: 74 CAAGGCTCAATTCATTCAAATT |
| Hlgaf | SEQ ID NO: 75 CCAATCAGCGCCATCAATC |
| Hlgar | SEQ ID NO: 76 CCAGTTGGGTCTTGTGCAAAT |
| Hlgbf | SEQ ID NO: 77 CGTTGCTACTTCTATGGCAT |
| Hlgbr | SEQ ID NO: 78 ACATTGTATTTAGCTCCCCAA |
| Hlgcf | SEQ ID NO: 79 ATGTTAAAGCTATGCGATGGCC |
| Hlgcr | SEQ ID NO: 80 AAGAGGTGGTAACTCACTGTCTGGA |
| Half | SEQ ID NO: 81 TGTTTGTTGTTTGGATGCTTTTCT |
| Hlar | SEQ ID NO: 82 GGTTTAGCCTGGCCTTCAGC |
| Hlbf | SEQ ID NO: 83 CACCTGTACTCGGCCGTTCT |
| Hlbr | SEQ ID NO: 84 TATACATCCCATGGCTTAGGTTTTTC |
| Segf | SEQ ID NO: 85 GGTAACAATCGACAATAGACAATCACTT |
| Segr | SEQ ID NO: 86 TCCAGATTCAAATGCAGAACCAT |
| Senf | SEQ ID NO: 87 TGGACTGTATTTTGGAAATAAATGTGT |
| Senr | SEQ ID NO: 88 GCTCCCACTGAACCTTTTACGT |
| Seuf | SEQ ID NO: 89 AGCGAGTGAATTCTCTGGTTTAATG |
| Seur | SEQ ID NO: 90 TTGTGCTGTTATGTTTTTCATATTGG |
| Seif | SEQ ID NO: 91 TGGCATTGATTATAATGGTCCTTG |
| Seir | SEQ ID NO: 92 GCCTTTACCAGTGTTATTATGACC |
| Semf | SEQ ID NO: 93 TCAGTTTCGACAGTTTTGTTGT |
| Semr | SEQ ID NO: 94 |
| | CAGCTCAAGAAATTGATACTAAATTAAGAAG |
| Seof | SEQ ID NO: 95 ACTACAGATAAAAAGAAAGTTACTGCAC |
| Seor | SEQ ID NO: 96 CATCAATATGATAGTCTGATGAATCTATTG |
| Secf | SEQ ID NO: 97 CAACCAGACCCTACGCCAGA |
| Secr | SEQ ID NO: 98 TGTTATAAATTAAATCATGTGCCAAAAA |
| Self | SEQ ID NO: 99 TAATTATCAATGGCAAGCATCAAAC |
| Selr | SEQ ID NO: 100 CACTCCCCTTATCAAAACCGC |
| Tstf | SEQ ID NO: 101 TGAATTTTTTTATCGTAAGCCCTTTG |
| Tstr | SEQ ID NO: 102 GGAAATGGATATAAGTTCCTTCGCT |
| Selxf | SEQ ID NO: 103 TCAACACAAAATTCCTCAAGTGT |
| Selxr | SEQ ID NO: 104 GCGACTCTAATGTATATTTACCGCC |

qRT-PCR

| | |
|---|---|
| Qintf | SEQ ID NO: 105 CATCACTGGTGGACGCTTTG |
| Qintr | SEQ ID NO: 106 AATGCATCGAGCGCTTTTTC |
| qcas9f | SEQ ID NO: 107 CGGAAGCGACTCGTCTCAA |
| qcas9r | SEQ ID NO: 108 CAAATACGATTCTTCCGACGTGTAT |
| qϕ11intf | SEQ ID NO: 109 TCTTTCTGTTGACTATGCACGATCT |
| qϕ11intr | SEQ ID NO: 110 TTTTGGCGTAATTGATAACTGCTT |
| qϕnm1intf | SEQ ID NO: 111 TTCTGTTGGCTATGCACGATCT |
| qϕnm1intr | SEQ ID NO: 112 TTTTGGCGTAATTGATAACTGCTT |

Allelic exchange construct. Integration of CRISPR-Cas9 system into the genome of ϕSaBov and marker-less deletion mutants of the nuc gene and 19 virulence genes were generated by allelic exchange using modified pMAD-secY temperature sensitive shuttle vector system by introducing a new multi-cloning site, a GFPuv reporter gene, a chloramphenicol resistant gene (cat), and an anti-sense secY gene controlled by a tetracycline inducible promoter into the pMAD system (Arnaud et al., 2004) to improve screening process of allelic exchange, as shown in FIG. 7. For an efficient screening process of double-crossover event, genes encoding chloramphenicol resistance (cat), anti-sense-secY controlled by teteracycline inducible promoter (secY), green fluorescent protein UV variant (GFUuv), and a new multi-cloning site were added to the original pMAD system.

Briefly, upstream and downstream fragments of target gene were amplified and cloned in modified pMAD-secY system in E. coli, followed by electroporation into S. aureus strains. The first homologous recombination was induced by culturing at 43° C. (non-permissive temperature for the replication of pMAD-secY), followed by culturing at 37° C. to promote the second recombination, resulting in allelic exchange. The mutant candidates were screened by growth in TSA plate supplemented with anhydrous tetracycline (0.5 μg/ml), loss of GFP expression, and no growth in TSA plate supplemented with chloramphenicol, indicating the second recombination.

Phage lysates. Phages were induced from the mid-exponential culture of strains by adding mitomycin C (1 μg/mL, Sigma-Aldrich) which induced clear lysis typically in 3 hours incubation at 30° C. with 80 rpm. The lysates were sterilized with syringe filers (0.22 p.m, Nalgene). Phage lysates were generated by propagating phage to the mid-exponential culture of the same strains from which phages were initially induced, followed by filter sterilization of lysates. The number of transducing phage particles (TP) was determined by calculating the plaque-forming unit using soft agar (0.5%) overlay method or quantitative real time PCR. Briefly, phage lysates were treated with excessive Dnase I (Sigma-Aldrich) to remove chromosomal DNA contamination, followed by DNA extraction from phage particles using DNeasy kit (Qiagen) as described previously (Moon et al., 2016, Moon et al., 2015). Quantitative real time PCR reaction was performed using SYBR green I master mix (Applied Biosystems), primer sets specific to phages, and a serial dilution of phage DNA templates. The absolute copy number of phage DNA was calculate by interpolation of the threshold cycle from phage DNA template to the standard curves generated from cloned plasmid templates.

In vitro efficacy tests. The mid exponential culture of recipient strains was harvested by centrifugation and adjusted to 1×10⁶ CFU/mL in PBS. A test tube killing assay was set up in 1 mL of reaction mixtures consisting of 100 μL of recipient cell suspension, 20 μL of serially diluted phage lysates, and 880 μL TSB, and incubated at 37° C. The number of viable cells at each time point was determined by serial dilution and plating onto TSA plates. For in vitro killing under nutritionally limited condition, an empty antibiotic disc was placed in sterile petri dish and inoculated with 100 μL of recipient cell suspension in PBS (1×10⁵ CFU), followed by 20 μL of serially diluted phage lysates. After 8 hour incubation at 37° C., the viable cells were recovered by blotting the disc onto TSA plates.

In vivo efficacy tests. All animal experiments were performed in compliance with a protocol reviewed and approved by the Institutional Animal Care and Use Committee at the Mississippi State University (14-040). The back of C57BL/6 mice (6 to 8 week old, female, Harlan laboratory) were shaved with electric razor, depilated with Nair cream, and decontaminated with 70% ethanol swab. For intradermal infection, 100 μl of bacterial suspension in PBS containing 1×10⁵ CFU was intradermally injected to the shaved skin. After 6 h, 100 μl of phage stock containing 5×10⁷ transducing phage particles was intradermally injected to the infected skin. For skin surface infection, shaved skin was topically infected with 5 μL of bacterial suspension containing 2×10⁴ CFU. After 1 h, 10 μL of phage stock containing 1×10⁷ transducing phage particles was applied to the infected skin. After 24 h, mice were euthanized with CO2, and infected skin was excised and homogenized using Omni TH tissue homogenizer (OMNI international). Homogenates were serially diluted and plated on to BHI plate to determine the number of viable cells.

Toxin detection in phage lysates. Heparinized human venous blood was collected from healthy volunteers. Written consent was obtained from each volunteer in compliance with a protocol reviewed and approved by the Institutional Review Board for Human Subjects at the Mississippi State University (12-041). Peripheral blood mononuclear cells (PBMCs) were isolated by density gradient centrifugation using Ficoll-Histopaque (Sigma-Aldrich). Purified PBMCs were adjusted to 2×10⁶ cell per well in 96 well cell culture plate in RPMI1640 medium supplemented with 10% FBS. Phage lysates prepared from RF122Δnuc or RF122-19Δnuc was added to the wells. To detect superantigens in phage lysates, proliferation of T cell was measured using a [³H]-thymidine incorporation assay as described previously (Seo et al., 2007). To detect cytotoxins in phage lysates, cytotoxicity of cells were measured using propidium iodide incorporation assay using LIVE/DEAD Cell-mediated Cytotoxicity kit (ThermoFisher).

### REFERENCES

1 Suaya, J. A. et al. Incidence and cost of hospitalizations associated with Staphylococcus aureus skin and soft tissue infections in the United States from 2001 through 2009. BMC infectious diseases 14, 296, doi:10.1186/1471-2334-14-296 (2014).
2 Klevens, R. M. et al. Invasive methicillin-resistant Staphylococcus aureus infections in the United States. Jama 298, 1763-1771, doi:10.1001/jama.298.15.1763 (2007). Hafer, C., Lin, Y., Kornblum, J., Lowy, F. D. & Uhlemann, A. C. Contribution of selected gene mutations to resistance in clinical isolates of vancomycin-intermediate Staphylococcus aureus. Antimicrobial agents and chemotherapy 56, 5845-5851, doi:10.1128/aac.01139-12 (2012).
4 Levine, D. P. Vancomycin: understanding its past and preserving its future. Southern medical journal 101, 284-291, doi:10.1097/SMJ.0b013e3181647037 (2008).
5 Weigel, L. M. et al. Genetic analysis of a high-level vancomycin-resistant isolate of Staphylococcus aureus. Science (New York, N.Y.) 302, 1569-1571, doi:10.1126/science.1090956 (2003).
6 Brown, E. D. & Wright, G. D. Antibacterial drug discovery in the resistance era. Nature 529, 336-343, doi:10.1038/nature17042 (2016).
7 Sorek, R., Lawrence, C. M. & Wiedenheft, B. CRISPR-mediated adaptive immune systems in bacteria and archaea. Annual review of biochemistry 82, 237-266, doi:10.1146/annurev-biochem-072911-172315 (2013).
8 Barrangou, R. et al. CRISPR provides acquired resistance against viruses in prokaryotes. Science (New York, N.Y.) 315, 1709-1712, doi:10.1126/science.1138140 (2007).
9 Mali, P., Esvelt, K. M. & Church, G. M. Cas9 as a versatile tool for engineering biology. Nature methods 10, 957-963, doi:10.1038/nmeth.2649 (2013).
10 Semenova, E. et al. Interference by clustered regularly interspaced short palindromic repeat (CRISPR) RNA is governed by a seed sequence. Proceedings of the National Academy of Sciences of the United States of America 108, 10098-10103, doi:10.1073/pnas.1104144108 (2011).
11 Mojica, F. J., Diez-Villasenor, C., Garcia-Martinez, J. & Almendros, C. Short motif sequences determine the targets of the prokaryotic CRISPR defence system. Microbiology (Reading, England) 155, 733-740, doi:10.1099/mic.0.023960-0 (2009).
12 Citorik, R. J., Mimee, M. & Lu, T. K. Sequence-specific antimicrobials using efficiently delivered RNA-guided nucleases. Nature biotechnology 32, 1141-1145, doi:10.1038/nbt.3011 (2014).
13 Yosef, I., Manor, M., Kiro, R. & Qimron, U. Temperate and lytic bacteriophages programmed to sensitize and kill antibiotic-resistant bacteria. Proceedings of the National Academy of Sciences of the United States of America 112, 7267-7272, doi:10.1073/pnas.1500107112 (2015).
14 Bikard, D. et al. Exploiting CRISPR-Cas nucleases to produce sequence-specific antimicrobials. Nature biotechnology 32, 1146-1150, doi:10.1038/nbt.3043 (2014).
15 Gomaa, A. A. et al. Programmable removal of bacterial strains by use of genome-targeting CRISPR-Cas systems. mBio 5, e00928-00913, doi:10.1128/mBio.00928-13 (2014).
16 Jiang, W., Bikard, D., Cox, D., Zhang, F. & Marraffini, L. A. RNA-guided editing of bacterial genomes using CRISPR-Cas systems. Nature biotechnology 31, 233-239, doi:10.1038/nbt.2508 (2013).
17 Penades, J. R., Chen, J., Quiles-Puchalt, N., Carpena, N. & Novick, R. P. Bacteriophage-mediated spread of bacterial virulence genes. Current opinion in microbiology 23, 171-178, doi:10.1016/j.mib.2014.11.019 (2015).
18 Ubeda, C. et al. Antibiotic-induced SOS response promotes horizontal dissemination of pathogenicity island-encoded virulence factors in staphylococci. Molecular microbiology 56, 836-844, doi:10.1111/j.1365-2958.2005.04584.x (2005).
19 Chen, J. & Novick, R. P. Phage-mediated intergeneric transfer of toxin genes. Science (New York, N.Y.) 323, 139-141, doi:10.1126/science.1164783 (2009).
20 Loc-Carrillo, C. & Abedon, S. T. Pros and cons of phage therapy. Bacteriophage 1, 111-114, doi:10.4161/bact.1.2.14590 (2011).
21 Bakhshinejad, B. & Sadeghizadeh, M. Bacteriophages as vehicles for gene delivery into mammalian cells: prospects and problems. Expert opinion on drug delivery 11, 1561-1574, doi:10.1517/17425247.2014.927437 (2014).
22 Pirnay, J. P. et al. Quality and safety requirements for sustainable phage therapy products. Pharmaceutical research 32, 2173-2179, doi:10.1007/s11095-014-1617-7 (2015).
23 Moon, B. Y. et al. Mobilization of Genomic Islands of Staphylococcus aureus by Temperate Bacteriophage. PloS one 11, e0151409, doi:10.1371/journal.pone.0151409 (2016).
24 Moon, B. Y. et al. Phage-mediated horizontal transfer of a Staphylococcus aureus virulence-associated genomic island. Scientific reports 5, 9784, doi:10.1038/srep09784 (2015).
25 Bartual, S. G. et al. Structure of the bacteriophage T4 long tail fiber receptor-binding tip. Proceedings of the National Academy of Sciences of the United States of America 107, 20287-20292, doi:10.1073/pnas.1011218107 (2010).
26 Winstel, V. et al. Wall teichoic acid structure governs horizontal gene transfer between major bacterial pathogens. Nature communications 4, 2345, doi:10.1038/ncomms3345 (2013).
27 Le, S. et al. Mapping the tail fiber as the receptor binding protein responsible for differential host specificity of Pseudomonas aeruginosa bacteriophages PaP1 and JG004. PloS one 8, e68562, doi:10.1371/journal.pone.0068562 (2013).
28 Beisel, C. L., Gomaa, A. A. & Barrangou, R. A CRISPR design for next-generation antimicrobials. Genome biology 15, 516, doi:10.1186/s13059-014-0516-x (2014).
29 Abremski, K. & Gottesman, S. Purification of the bacteriophage lambda xis gene product required for lambda excisive recombination. The Journal of biological chemistry 257, 9658-9662 (1982).
30 Ball, C. A. & Johnson, R. C. Multiple effects of Fis on integration and the control of lysogeny in phage lambda. Journal of bacteriology 173, 4032-4038 (1991).
31 Iandolo, J. J. et al. Comparative analysis of the genomes of the temperate bacteriophages phi 11, phi 12 and phi 13 of Staphylococcus aureus 8325. Gene 289, 109-118 (2002).
32 Herron-Olson, L., Fitzgerald, J. R., Musser, J. M. & Kapur, V. Molecular correlates of host specialization in Staphylococcus aureus. PloS one 2, el120, doi: 10.1371/journal.pone.0001120 (2007).
33 Perera, M. N., Abuladze, T., Li, M., Woolston, J. & Sulakvelidze, A. Bacteriophage cocktail significantly reduces or eliminates Listeria monocytogenes contamination on lettuce, apples, cheese, smoked salmon and frozen foods. Food microbiology 52, 42-48, doi:10.1016/j.fm.2015.06.006 (2015).
34 Chen, J., Ram, G., Penades, J. R., Brown, S. & Novick, R. P. Pathogenicity island-directed transfer of unlinked chromosomal virulence genes. Molecular cell 57, 138-149, doi:10.1016/j.molce1.2014.11.011 (2015).
35 Arnaud, M., Chastanet, A. & Debarbouille, M. New vector for efficient allelic replacement in naturally nontransformable, low-GC-content, gram-positive bacteria. Applied and environmental microbiology 70, 6887-6891, doi:10.1128/aem.70.11.6887-6891.2004 (2004)
36 Seo, K. S. et al. Long-term staphylococcal enterotoxin C1 exposure induces soluble factor-mediated immunosuppression by bovine CD4+ and CD8+ T cells. Infection and immunity 75, 260-269, doi:10.1128/iai.01358-06 (2007).
37 Joo Y S, Fox L K, Davis W C, Bohach G A, & Park Y H (2001) Staphylococcus aureus associated with mammary glands of cows: genotyping to distinguish different strains among herds. Veterinary microbiology 80(2):131-138.
38. McDougal L K, et al. (2003) Pulsed-field gel electrophoresis typing of oxacillin-resistant Staphylococcus aureus isolates from the United States: establishing a national database. Journal of clinical microbiology 41(11):5113-5120.
39. Baba T, et al. (2002) Genome and virulence determinants of high virulence community-acquired MRSA. Lancet (London, England) 359(9320): 1819-1827.
40. Sullivan M A, Yasbin R E, & Young F E (1984) New shuttle vectors for Bacillus subtilis and Escherichia coli which allow rapid detection of inserted fragments. Gene 29(1-2):21-26.

I claim:

1. A method for the production of sequence specific antimicrobials to improve delivery to target cells comprising:
a. programming a Cas9 nuclease to specific oligonucleotide sequence via a pKS1 plasmid,
b. integrating CRISPR/Cas9 system into the genome of a temperate phage by allelic exchange via a pKS4 plasmid, and
c. expanding host specificity of phage by complementing a phage tail fiber protein.

2. The allelic exchange of claim 1 wherein said allelic exchange comprises using a modified pMAD-secY shuttle vector system comprising introducing a new multi-cloning site, a green fluorescent protein UV variant reporter gene (GFUuv), a chloramphenicol resistant gene (cat), and an anti-sense secY gene controlled by a tetracycline inducible promoter into a modified pMAD system.

3. The allelic exchange of claim 2, wherein the pMAD-secY system is temperature sensitive.

4. A method of using CRISPR/Cas9 as an antimicrobial comprising:
a. induction and amplification of a temperate phages harboring CRISPR/Cas9 system in the phage genome,
b. removing virulence genes from the host chromosome to prevent contamination of toxins in phage lysates, and
c. expanding host specificity of phage by complementing a phage tail fiber protein.

5. The allelic exchange of claim 4 wherein said allelic exchange comprises using a modified pMAD-secY shuttle vector system comprising introducing a new multi-cloning site, a green fluorescent protein UV variant reporter gene (GFUuv), a chloramphenicol resistant gene (cat), and an anti-sense secY gene controlled by a tetracycline inducible promoter into a modified pMAD system.

6. The allelic exchange of claim 5, wherein the pMAD-secY system is temperature sensitive.

7. The CRISPR/Cas9 antimicrobials of claim 4 wherein said CRISPR/Cas9 antimicrobials are medicaments.

8. The medicaments of claim 7 wherein said medicaments are antibiotics.

9. The medicaments of claim 7 wherein said medicament are applied topically to infected tissues.

10. The CRIPSR/Cas9 antimicrobials of claim 4 wherein the CRIPSR/Cas9 antimicrobials sanitize the contaminated surface of medical devices.

11. The CRIPSR/Cas9 antimicrobials of claim 4 wherein the CRIPSR/Cas9 antimicrobials sanitize the contaminated surface of culinary devices.

12. The CRIPSR/Cas9 antimicrobials of claim 4 wherein the CIRPSR/Cas9 antimicrobials sanitize the contaminated surface of food products.

13. The phage lysates of claim 1 wherein said phase lysates further comprise mixtures of bacterial components including bacterial DNA, proteins, and cell wall components, as well as transducing phage particles.

14. An improved method of allelic exchange using a modified pMAD-secY shuttle vector system comprising introducing a new multi-cloning site, a green fluorescent protein UV variant reporter gene (GFUuv), a chloramphenicol resistant gene (cat), and an anti-sense secY gene controlled by a tetracycline inducible promoter into a modified pMAD system, wherein the pMAD-secY system is temperature sensitive.

15. A pKS1 plasmid for generating a programmable CRISPR/Cas9 system comprising synthetic oligos, wherein said synthetic oligos further comprise a CRISPR array encoding promoter, pre-crRNA, and direct repeats interspaced with two Bbsl restriction sites cloned into pMK4.

16. A method of using a pKS1 plasmid to generate a programmable CRIPSR/Cas9 system comprising cloning synthetic oligonucleotides to program the target of Cas9 nuclease and transcribing precrRNA that guides Cas9 to the specific target sequences.

17. A pKS4 plasmid for generating a programmable CRISPR/Cas9 system comprising a cloned CRISPR array with a spacer sequence specific to the nuc in pKS2 cloned into pKS3.

18. The pKS5 plasmid of claim 17 wherein the programmable CRIPSR/Cas9 system is specific to Staphylococcus aureus.

19. A method of using a pKS4 plasmid to generate a programmable CRIPSR/Cas9 system comprising:
a. transcribing tracRNA and Cas9 nuclease,
b. cloning and transcribing precrRNA,
c. temperature sensitive replication for spontaneous curing,
d. multi-cloning sites for homologous recombinations, and
e. expressing green fluorescence protein to rapid screening process.


SEQUENCE LISTING (112 sequences)

SEQ ID NO: 1 - DNA, 294 bp, Artificial Sequence
  Description of Artificial Sequence Synthetic polynucleotide
gatcccggccgacgtgaactatatgattttccgcagtatattttagatgaagattatttcttaataactaaaaatatggtataatactcttaataaatgcagtaatacaggggcttttcaagactgaagtctagctgagacaaatagtgcgattacgaaattttttagacaaaaatagtctacgaggttttagagctatgctgttttgaatggtcccaaaacgggtcttcgatcgatcgatcgaagacgttttagagctatgctgttttgaatggtcccaaaaccttgcagggg

SEQ ID NO: 2 - DNA, 294 bp, Artificial Sequence
  Description of Artificial Sequence Synthetic polynucleotide
aattcccctgcagggttttgggaccattcaaaacagcatagctctaaaacgtcttcgatcgatcgatcgaagacccgttttgggaccattcaaaacagcatagctctaaaacctcgtagactatttttgtctaaaaaatttcgtaatcgcactatttgtctcagctagacttcagtcttgaaaagcccctgtattactgcatttattaagagtattataccatatttttagttattaagaaataatcttcatctaaaatatactgcggaaaatcatatagttcacgtcggccgg

SEQ ID NO: 3 - DNA, 36 bp, Artificial Sequence
  Description of Artificial Sequence Synthetic oligonucleotide
aaactgcaaagaaaattgaagtcgagtttgacaagt

SEQ ID NO: 4 - DNA, 36 bp, Artificial Sequence
  Description of Artificial Sequence Synthetic oligonucleotide
taaaacttgtcaaactcgacttcaattttctttgca

SEQ ID NO: 5 - DNA, 31 bp, Artificial Sequence
  Description of Artificial Sequence Synthetic oligonucleotide
gatccttaagtgatcccttgaaagattctgt

SEQ ID NO: 6 - DNA, 33 bp, Artificial Sequence
  Description of Artificial Sequence Synthetic oligonucleotide
gatccggccgtcagtcacctcctagctgactca

SEQ ID NO: 7 - DNA, 45 bp, Artificial Sequence
  Description of Artificial Sequence Synthetic oligonucleotide
gcaggtcgacggatcccggccgacgtgaactatatgattttccgc

SEQ ID NO: 8 - DNA, 90 bp, Artificial Sequence
  Description of Artificial Sequence Synthetic oligonucleotide
aacgacggccagtgaattcccctgcagggttttgggaccattcaaaacagcatagctctaaaacgtcttcgatcgatcgatcgaagaccc

SEQ ID NO: 9 - DNA, 29 bp, Artificial Sequence
  Description of Artificial Sequence Synthetic oligonucleotide
gatcgtcgacttatgcttcactccatttc

SEQ ID NO: 10 - DNA, 30 bp, Artificial Sequence
  Description of Artificial Sequence Synthetic oligonucleotide
gatccttaagatgggcagtgttgtaattat

SEQ ID NO: 11 - DNA, 32 bp, Artificial Sequence
  Description of Artificial Sequence Synthetic oligonucleotide
gatccctgcaggtgttgttgcattaaatcact

SEQ ID NO: 12 - DNA, 30 bp, Artificial Sequence
  Description of Artificial Sequence Synthetic oligonucleotide
gcgcagatcttgatatttagaggtggcaca

SEQ ID NO: 13 - DNA, 35 bp, Artificial Sequence
  Description of Artificial Sequence Synthetic oligonucleotide
gatcgtcgacgttaacactttaagcaaaccgcatc

SEQ ID NO: 14 - DNA, 33 bp, Artificial Sequence
  Description of Artificial Sequence Synthetic oligonucleotide
gatcacgcgtaactaacacctctttctttttag

SEQ ID NO: 15 - DNA, 35 bp, Artificial Sequence
  Description of Artificial Sequence Synthetic oligonucleotide
gatcgaattctgctcattgtaaaagtgtcactgct

SEQ ID NO: 16 - DNA, 30 bp, Artificial Sequence
  Description of Artificial Sequence Synthetic oligonucleotide
gatccccgggatacgtcgctaccatcttct

SEQ ID NO: 17 - DNA, 30 bp, Artificial Sequence
  Description of Artificial Sequence Synthetic oligonucleotide
gcgcggatccttacctcatatagtgtcatg

SEQ ID NO: 18 - DNA, 30 bp, Artificial Sequence
  Description of Artificial Sequence Synthetic oligonucleotide
gcgcgtcgacgaaaggtaccattgctggtc

SEQ ID NO: 19 - DNA, 30 bp, Artificial Sequence
  Description of Artificial Sequence Synthetic oligonucleotide
gcgcgaattcgtcaatttagaatattgcag

SEQ ID NO: 20 - DNA, 30 bp, Artificial Sequence
  Description of Artificial Sequence Synthetic oligonucleotide
gcgcagatctaatgcctctaactaaaaacc

SEQ ID NO: 21 - DNA, 30 bp, Artificial Sequence
  Description of Artificial Sequence Synthetic oligonucleotide
gcgcggatcccttaattccgattacatttg

SEQ ID NO: 22 - DNA, 30 bp, Artificial Sequence
  Description of Artificial Sequence Synthetic oligonucleotide
gcgcgtcgacgtgcctttattaacattaag

SEQ ID NO: 23 - DNA, 30 bp, Artificial Sequence
  Description of Artificial Sequence Synthetic oligonucleotide
gcgcgaattccttcaagtcattcgcaataa

SEQ ID NO: 24 - DNA, 30 bp, Artificial Sequence
  Description of Artificial Sequence Synthetic oligonucleotide
gcgcagatctgtatcaacgatcttattaac

SEQ ID NO: 25 - DNA, 28 bp, Artificial Sequence
  Description of Artificial Sequence Synthetic oligonucleotide
taatggatccaccgttgattctcaatcg

SEQ ID NO: 26 - DNA, 28 bp, Artificial Sequence
  Description of Artificial Sequence Synthetic oligonucleotide
tgaagtcgaccatcttaacaactagggc

SEQ ID NO: 27 - DNA, 30 bp, Artificial Sequence
  Description of Artificial Sequence Synthetic oligonucleotide
gcgcgaattcggctttgtgaaacctaatcc

SEQ ID NO: 28 - DNA, 30 bp, Artificial Sequence
  Description of Artificial Sequence Synthetic oligonucleotide
gcgcagatctggtcgtcacaattactgttg

SEQ ID NO: 29 - DNA, 29 bp, Artificial Sequence
  Description of Artificial Sequence Synthetic oligonucleotide
gcgcggatcccgaatttggagatggctgc

SEQ ID NO: 30 - DNA, 30 bp, Artificial Sequence
  Description of Artificial Sequence Synthetic oligonucleotide
gcgcgtcgacctaatcctggagtataactg

SEQ ID NO: 31 - DNA, 28 bp, Artificial Sequence
  Description of Artificial Sequence Synthetic oligonucleotide
tgatgaattcctattgcccgttaaacgg

SEQ ID NO: 32 - DNA, 29 bp, Artificial Sequence
  Description of Artificial Sequence Synthetic oligonucleotide
attgagatctcctgtcggtttactcattg

SEQ ID NO: 33 - DNA, 28 bp, Artificial Sequence
  Description of Artificial Sequence Synthetic oligonucleotide
gcgcggatccttcgtataggctttatag

SEQ ID NO: 34 - DNA, 28 bp, Artificial Sequence
  Description of Artificial Sequence Synthetic oligonucleotide
gcgcgtcgacctccaatgttatatccta

SEQ ID NO: 35 - DNA, 28 bp, Artificial Sequence
  Description of Artificial Sequence Synthetic oligonucleotide
gcgcgaattcctacttcctagataccgt

SEQ ID NO: 36 - DNA, 28 bp, Artificial Sequence
  Description of Artificial Sequence Synthetic oligonucleotide
gcgcagatctgaatagcttaacaacgta

SEQ ID NO: 37 - DNA, 24 bp, Artificial Sequence
  Description of Artificial Sequence Synthetic oligonucleotide
tcttgatacgtatttgacacttgc

SEQ ID NO: 38 - DNA, 24 bp, Artificial Sequence
  Description of Artificial Sequence Synthetic oligonucleotide
agctatacgagtttgatggttctg

SEQ ID NO: 39 - DNA, 48 bp, Artificial Sequence
  Description of Artificial Sequence Synthetic oligonucleotide
cagaaccatcaaactcgtatagctaactaagcgactcagataatagac

SEQ ID NO: 40 - DNA, 23 bp, Artificial Sequence
  Description of Artificial Sequence Synthetic oligonucleotide
agagttgttacagtcgctacacc

SEQ ID NO: 41 - DNA, 28 bp, Artificial Sequence
  Description of Artificial Sequence Synthetic oligonucleotide
atgaattcctgtggatttagaaataagg

SEQ ID NO: 42 - DNA, 21 bp, Artificial Sequence
  Description of Artificial Sequence Synthetic oligonucleotide
ccaacattcccaagaagtatc

SEQ ID NO: 43 - DNA, 45 bp, Artificial Sequence
  Description of Artificial Sequence Synthetic oligonucleotide
gatacttcttcttgggaatgttggaagaatggataatgttaatcc

SEQ ID NO: 44 - DNA, 22 bp, Artificial Sequence
  Description of Artificial Sequence Synthetic oligonucleotide
ttatccatggcaagcatcaaac

SEQ ID NO: 45 - DNA, 26 bp, Artificial Sequence
  Description of Artificial Sequence Synthetic oligonucleotide
gatatatttgaaaggtaagtacttcg

SEQ ID NO: 46 - DNA, 27 bp, Artificial Sequence
  Description of Artificial Sequence Synthetic oligonucleotide
agtgtagtattccatatgaatgatggt

SEQ ID NO: 47 - DNA, 51 bp, Artificial Sequence
  Description of Artificial Sequence Synthetic oligonucleotide
accatcattcatatggaatactacactatacaaaaggttataggaagagtt

SEQ ID NO: 48 - DNA, 23 bp, Artificial Sequence
  Description of Artificial Sequence Synthetic oligonucleotide
caatttctacagatatgactccc

SEQ ID NO: 49 - DNA, 20 bp, Artificial Sequence
  Description of Artificial Sequence Synthetic oligonucleotide
gtcatgtttcggttgatagg

SEQ ID NO: 50 - DNA, 26 bp, Artificial Sequence
  Description of Artificial Sequence Synthetic oligonucleotide
tgtacaaatggacttaagatatagcg

SEQ ID NO: 51 - DNA, 32 bp, Artificial Sequence
  Description of Artificial Sequence Synthetic oligonucleotide
gcgcggatccactacatgtacttaccaatgcg

SEQ ID NO: 52 - DNA, 40 bp, Artificial Sequence
  Description of Artificial Sequence Synthetic oligonucleotide
gcgcgtcgacgcagaaattaattaatttaccactttttct

SEQ ID NO: 53 - DNA, 37 bp, Artificial Sequence
  Description of Artificial Sequence Synthetic oligonucleotide
gcgcgaattccaaagggcttacgatgaaaaatttcat

SEQ ID NO: 54 - DNA, 34 bp, Artificial Sequence
  Description of Artificial Sequence Synthetic oligonucleotide
gcgcagatctaaccaattaccaaattctccatgc

SEQ ID NO: 55 - DNA, 23 bp, Artificial Sequence
  Description of Artificial Sequence Synthetic oligonucleotide
tgtcgatgctatggatagtgagg

SEQ ID NO: 56 - DNA, 23 bp, Artificial Sequence
  Description of Artificial Sequence Synthetic oligonucleotide
taattacctccttgatgtaaagc

SEQ ID NO: 57 - DNA, 48 bp, Artificial Sequence
  Description of Artificial Sequence Synthetic oligonucleotide
gctttacatcaaggaggtaattatatcgctaatactttgaaagttagg

SEQ ID NO: 58 - DNA, 28 bp, Artificial Sequence
  Description of Artificial Sequence Synthetic oligonucleotide
tcaaatgtagcagtatacattaattgcg

SEQ ID NO: 59 - DNA, 30 bp, Artificial Sequence
  Description of Artificial Sequence Synthetic oligonucleotide
gctactgcagatcccattatgctttggcag

SEQ ID NO: 60 - DNA, 21 bp, Artificial Sequence
  Description of Artificial Sequence Synthetic oligonucleotide
gggtttcactctccttctaca

SEQ ID NO: 61 - DNA, 41 bp, Artificial Sequence
  Description of Artificial Sequence Synthetic oligonucleotide
aggagagtgaaacccatgtacaaaataaaagatgttgaaac

SEQ ID NO: 62 - DNA, 32 bp, Artificial Sequence
  Description of Artificial Sequence Synthetic oligonucleotide
gctaggatccctaactgatttctccccataag

SEQ ID NO: 63 - DNA, 27 bp, Artificial Sequence
  Description of Artificial Sequence Synthetic oligonucleotide
ttgccagtcaacttcataagtagatgt

SEQ ID NO: 64 - DNA, 20 bp, Artificial Sequence
  Description of Artificial Sequence Synthetic oligonucleotide
gcgcgtggtaactttaaccc

SEQ ID NO: 65 - DNA, 22 bp, Artificial Sequence
  Description of Artificial Sequence Synthetic oligonucleotide
ttttttaccatcaggcgtaaca

SEQ ID NO: 66 - DNA, 20 bp, Artificial Sequence
  Description of Artificial Sequence Synthetic oligonucleotide
acgaatgatttggccattcc

SEQ ID NO: 67 - DNA, 22 bp, Artificial Sequence
  Description of Artificial Sequence Synthetic oligonucleotide
tggaggtaaattccagtcagca

SEQ ID NO: 68 - DNA, 21 bp, Artificial Sequence
  Description of Artificial Sequence Synthetic oligonucleotide
tgtcgcgataaaagacggatt

SEQ ID NO: 69 - DNA, 20 bp, Artificial Sequence
  Description of Artificial Sequence Synthetic oligonucleotide
actggtggattgaacgggtc

SEQ ID NO: 70 - DNA, 22 bp, Artificial Sequence
  Description of Artificial Sequence Synthetic oligonucleotide
atccagtgcaagttgttccaaa

SEQ ID NO: 71 - DNA, 24 bp, Artificial Sequence
  Description of Artificial Sequence Synthetic oligonucleotide
gactttgcaccaaaaaatcaggat

SEQ ID NO: 72 - DNA, 24 bp, Artificial Sequence
  Description of Artificial Sequence Synthetic oligonucleotide
aggtgcataatgtattccaggtct

SEQ ID NO: 73 - DNA, 22 bp, Artificial Sequence
  Description of Artificial Sequence Synthetic oligonucleotide
gcgtcatcattatcatgtgcaa

SEQ ID NO: 74 - DNA, 22 bp, Artificial Sequence
  Description of Artificial Sequence Synthetic oligonucleotide
caaggctcaattcattcaaatt

SEQ ID NO: 75 - DNA, 19 bp, Artificial Sequence
  Description of Artificial Sequence Synthetic oligonucleotide
ccaatcagcgccatcaatc

SEQ ID NO: 76 - DNA, 21 bp, Artificial Sequence
  Description of Artificial Sequence Synthetic oligonucleotide
ccagttgggtcttgtgcaaat

SEQ ID NO: 77 - DNA, 20 bp, Artificial Sequence
  Description of Artificial Sequence Synthetic oligonucleotide
cgttgctacttctatggcat

SEQ ID NO: 78 - DNA, 21 bp, Artificial Sequence
  Description of Artificial Sequence Synthetic oligonucleotide
acattgtatttagctccccaa

SEQ ID NO: 79 - DNA, 22 bp, Artificial Sequence
  Description of Artificial Sequence Synthetic oligonucleotide
atgttaaagctatgcgatggcc

SEQ ID NO: 80 - DNA, 25 bp, Artificial Sequence
  Description of Artificial Sequence Synthetic oligonucleotide
aagaggtggtaactcactgtctgga

SEQ ID NO: 81 - DNA, 24 bp, Artificial Sequence
  Description of Artificial Sequence Synthetic oligonucleotide
tgtttgttgtttggatgcttttct

SEQ ID NO: 82 - DNA, 20 bp, Artificial Sequence
  Description of Artificial Sequence Synthetic oligonucleotide
ggtttagcctggccttcagc

SEQ ID NO: 83 - DNA, 20 bp, Artificial Sequence
  Description of Artificial Sequence Synthetic oligonucleotide
cacctgtactcggccgttct

SEQ ID NO: 84 - DNA, 26 bp, Artificial Sequence
  Description of Artificial Sequence Synthetic oligonucleotide
tatacatcccatggcttaggtttttc

SEQ ID NO: 85 - DNA, 28 bp, Artificial Sequence
  Description of Artificial Sequence Synthetic oligonucleotide
ggtaacaatcgacaatagacaatcactt

SEQ ID NO: 86 - DNA, 23 bp, Artificial Sequence
  Description of Artificial Sequence Synthetic oligonucleotide
tccagattcaaatgcagaaccat

SEQ ID NO: 87 - DNA, 27 bp, Artificial Sequence
  Description of Artificial Sequence Synthetic oligonucleotide
tggactgtattttggaaataaatgtgt

SEQ ID NO: 88 - DNA, 22 bp, Artificial Sequence
  Description of Artificial Sequence Synthetic oligonucleotide
gctcccactgaaccttttacgt

SEQ ID NO: 89 - DNA, 25 bp, Artificial Sequence
  Description of Artificial Sequence Synthetic oligonucleotide
agcgagtgaattctctggtttaatg

SEQ ID NO: 90 - DNA, 26 bp, Artificial Sequence
  Description of Artificial Sequence Synthetic oligonucleotide
ttgtgctgttatgtttttcatattgg

SEQ ID NO: 91 - DNA, 24 bp, Artificial Sequence
  Description of Artificial Sequence Synthetic oligonucleotide
tggcattgattataatggtccttg

SEQ ID NO: 92 - DNA, 24 bp, Artificial Sequence
  Description of Artificial Sequence Synthetic oligonucleotide
gcctttaccagtgttattatgacc

SEQ ID NO: 93 - DNA, 22 bp, Artificial Sequence
  Description of Artificial Sequence Synthetic oligonucleotide
tcagtttcgacagttttgttgt

SEQ ID NO: 94 - DNA, 31 bp, Artificial Sequence
  Description of Artificial Sequence Synthetic oligonucleotide
cagctcaagaaattgatactaaattaagaag

SEQ ID NO: 95 - DNA, 28 bp, Artificial Sequence
  Description of Artificial Sequence Synthetic oligonucleotide
actacagataaaaagaaagttactgcac

SEQ ID NO: 96 - DNA, 30 bp, Artificial Sequence
  Description of Artificial Sequence Synthetic oligonucleotide
catcaatatgatagtctgatgaatctattg

SEQ ID NO: 97 - DNA, 20 bp, Artificial Sequence
  Description of Artificial Sequence Synthetic oligonucleotide
caaccagaccctacgccaga

SEQ ID NO: 98 - DNA, 28 bp, Artificial Sequence
  Description of Artificial Sequence Synthetic oligonucleotide
tgttataaattaaatcatgtgccaaaaa

SEQ ID NO: 99 - DNA, 25 bp, Artificial Sequence
  Description of Artificial Sequence Synthetic oligonucleotide
taattatcaatggcaagcatcaaac

SEQ ID NO: 100 - DNA, 21 bp, Artificial Sequence
  Description of Artificial Sequence Synthetic oligonucleotide
cactccccttatcaaaaccgc

SEQ ID NO: 101 - DNA, 26 bp, Artificial Sequence
  Description of Artificial Sequence Synthetic oligonucleotide
tgaatttttttatcgtaagccctttg

SEQ ID NO: 102 - DNA, 25 bp, Artificial Sequence
  Description of Artificial Sequence Synthetic oligonucleotide
ggaaatggatataagttccttcgct

SEQ ID NO: 103 - DNA, 23 bp, Artificial Sequence
  Description of Artificial Sequence Synthetic oligonucleotide
tcaacacaaaattcctcaagtgt

SEQ ID NO: 104 - DNA, 25 bp, Artificial Sequence
  Description of Artificial Sequence Synthetic oligonucleotide
gcgactctaatgtatatttaccgcc

SEQ ID NO: 105 - DNA, 20 bp, Artificial Sequence
  Description of Artificial Sequence Synthetic oligonucleotide
catcactggtggacgctttg

SEQ ID NO: 106 - DNA, 20 bp, Artificial Sequence
  Description of Artificial Sequence Synthetic oligonucleotide
aatgcatcgagcgctttttc

SEQ ID NO: 107 - DNA, 19 bp, Artificial Sequence
  Description of Artificial Sequence Synthetic oligonucleotide
cggaagcgactcgtctcaa

SEQ ID NO: 108 - DNA, 25 bp, Artificial Sequence
  Description of Artificial Sequence Synthetic oligonucleotide
caaatacgattcttccgacgtgtat

SEQ ID NO: 109 - DNA, 25 bp, Artificial Sequence
  Description of Artificial Sequence Synthetic oligonucleotide
tctttctgttgactatgcacgatct

SEQ ID NO: 110 - DNA, 24 bp, Artificial Sequence
  Description of Artificial Sequence Synthetic oligonucleotide
ttttggcgtaattgataactgctt

SEQ ID NO: 111 - DNA, 22 bp, Artificial Sequence
  Description of Artificial Sequence Synthetic oligonucleotide
ttctgttggctatgcacgatct

SEQ ID NO: 112 - DNA, 24 bp, Artificial Sequence
  Description of Artificial Sequence Synthetic oligonucleotide
ttttggcgtaattgataactgctt